(19) United States
(12) Patent Application Publication
(10) Pub. No.: US 2024/0327930 A1
(43) Pub. Date: Oct. 3, 2024

(54) COMPOSITIONS AND METHODS FOR DETECTING PROSTATE CANCER

(71) Applicant: IMMUNIS.AI, INC., Corvallis, OR (US)

(72) Inventors: Amin I. Kassis, Corvallis, OR (US); Geoffrey Erickson, Corvallis, OR (US); Kirk J. Wojno, Corvallis, OR (US); Leander Van Neste, Corvallis, OR (US); Ricardo Henao, Corvallis, OR (US); Harry Stylli, La Jolla, CA (US)

(21) Appl. No.: 18/681,604

(22) PCT Filed: Aug. 5, 2022
(86) PCT No.: PCT/US2022/039507

§ 371 Date: Feb. 6, 2024

Related U.S. Application Data
(60) Provisional application No. 63/230,509, filed on Aug. 6, 2021.

(51) Int. Cl. C12Q 1/6886; G01N 33/50; G01N 33/569; G01N 33/574

(52) U.S. Cl. CPC C12Q 1/6886; G01N 33/5047; G01N 33/56972; G01N 33/57434; C12Q 2600/118; C12Q 2600/158; G01N 2800/56

(57) ABSTRACT

The present disclosure relates to compositions and methods for assessing prostate cancer (e.g., identification of the aggressiveness or indolence of prostate cancer) in a subject. The compositions and methods include obtaining subject specific information (e.g., age, digital rectal exam (DRE) data, prostate volume, prostate density, total prostate-specific antigen (PSA)) and obtaining a biological sample from a subject and determining a measurement for a panel of biomarkers in the biological sample. Compositions and methods of the disclosure find use in both clinical and research settings, for example, within the fields of biology, immunology, medicine, and oncology.

### CROSS-REFERENCE TO RELATED APPLICATIONS

This application claims the benefit of U.S. Provisional Patent Application No. 63/230,509, filed Aug. 6, 2021, the contents of which are incorporated by reference herein.

### FIELD OF THE DISCLOSURE

The present disclosure relates to compositions and methods for assessing prostate cancer (e.g., identification of the aggressiveness or indolence of prostate cancer) in a subject. The compositions and methods include obtaining subject specific information (e.g., age, digital rectal exam (DRE) data, prostate density, total prostate-specific antigen (PSA)) and obtaining a biological sample from a subject and determining a measurement for a panel of biomarkers in the biological sample. Compositions and methods of the disclosure find use in both clinical and research settings, for example, within the fields of biology, immunology, medicine, and oncology.

### BACKGROUND

Prostate cancer is the second most common type of cancer and the fifth leading cause of cancer-related death in men (World Cancer Report 2014. World Health Organization. 2014). In 2012, it occurred in 1.1 million men and caused 307,000 deaths. It was the most common cancer in males in 84 countries (World Cancer Report 2014. World Health Organization. 2014. pp. Chapter 5.11), occurring more commonly in the developed world where rates of occurrence have been increasing.

Early diagnosis of prostate cancer often increases the likelihood of successful treatment or cure of such disease. Current diagnostic methods, however, depend largely on population-derived average values obtained from healthy individuals.

Personalized diagnostic methods are needed that enable the diagnosis, especially the early diagnosis, of the presence of prostate cancer in individuals who are not known to have the cancer or who have recurrent prostate cancer.

Leukocytes begin as pluripotent hematopoietic stem cells in the bone marrow and develop along either the myeloid lineage (monocytes, macrophages, neutrophils, eosinophils, and basophils) or the lymphoid lineage (T and B lymphocytes and natural killer cells). The major function of the myeloid lineage cells (e.g., neutrophils and macrophages) is the phagocytosis of infectious organisms, live unwanted damaged cells, senescent and dead cells (apoptotic and necrotic), as well as the clearing of cellular debris. Phagocytes from healthy animals do not replicate and are diploid, i.e., have a DNA content of 2n. On average, each cell contains <10 ng DNA, <20 ng RNA, and <300 ng of protein. Non-phagocytic cells are also diploid and are not involved in the internalization of dead cells or infectious organisms and have a DNA index of one.

The lifetime of various white blood cell subpopulations varies from a few days (e.g., neutrophils) to several months (e.g., macrophages). Like other cell types, leukocytes age and eventually die. During their aging process, human blood- and tissue-derived phagocytes (e.g., neutrophils) exhibit all the classic markers of programmed cell death (apoptosis), including caspase activation, pyknotic nuclei, and chromatin fragmentation. These cells also display a number of “eat-me” flags (e.g., phosphatidylserine, sugars) on the extracellular surfaces of their plasma membranes. Consequently, dying and dead cells and subcellular fragments thereof are cleared from tissues and blood by other phagocytic cells.

Although prostate-specific antigen (PSA) is considered an effective tumor marker and generally organ specific, it is not cancer specific. There is considerable overlap in PSA concentrations in men with prostate cancer and men with benign prostatic diseases. PSA does not differentiate men with organ confined prostate cancer (that may benefit from surgery) from those men with non-organ confined prostate cancer (that would not benefit from surgery). Therefore, PSA is not effective in selecting patients for radical prostatectomy.

While PSA is currently one of the most widely used diagnostic measures used to detect prostate cancer, false positives and false negatives are common, resulting in mistreatment of patients with no prostate cancer or overtreatment of patients with non-lethal prostate cancer. Improved methods for detecting prostate cancer are needed.

### SUMMARY

The present disclosure relates to compositions and methods for assessing prostate cancer (e.g., identification of the aggressiveness or indolence of prostate cancer) in a subject. Compositions and methods of the disclosure find use in the identification, characterization, and classification (e.g., via computing aggressiveness index) of cancer in a subject.

In some embodiments, the disclosure provides a method for identifying, assessing and/or predicting the aggressiveness or indolence of cancer (e.g., prostate cancer) in a subject (e.g., a subject suspected of having cancer, a subject diagnosed with a cancer, or a subject at risk for cancer). In some embodiments, the disclosure provides a method for identifying, assessing and/or predicting the aggressiveness or indolence of prostate cancer.

In some embodiments, the disclosure provides a method of measuring a panel of biomarkers in a subject comprising obtaining a biological sample from the subject; determining a measurement for the panel of biomarkers in the biological sample, wherein the panel of biomarkers comprise one or more (e.g., two, three, four, five, six, seven, eight, nine, ten, eleven, twelve, thirteen, fourteen, fifteen, or more) biomarkers selected from those shown in Table 1, below, and wherein the measurement comprises measuring a level of each of the biomarkers in the panel. In some embodiments, the panel of biomarkers comprises one or more (e.g., two, three, four, five, six, seven, eight, nine, ten, eleven, twelve, thirteen, fourteen, fifteen, or more) biomarkers selected from the clinical covariates shown in Table 1, and/or the genomic covariates listed in Table 1. In some embodiments, measuring the panel of biomarkers in the subject identifies, assesses, and/or predicts the aggressiveness or indolence of cancer (e.g., prostate cancer) in a subject (e.g., a subject suspected of having cancer, a subject diagnosed with a cancer, or a subject at risk for cancer). In some embodiments, the biological sample comprises CD2⁺ cells and/or CD14⁺ cells. In one embodiment, determining a measurement for the panel of biomarkers in the biological sample comprises measuring a level of each of the biomarkers in the panel in CD2⁺ cells and/or CD14⁺ cells. In one embodiment, the method further comprises obtaining one or more clinical data from the subject selected from the group consisting of age, race, digital rectal exam (DRE), prostate density, and total prostate-specific antigen (PSA). The disclosure is not limited by the type of clinical data obtained and/or used. Additional examples of clinical data include, but are not limited to, tumor stage, tumor grade, tumor size, tumor visual characteristics, tumor growth, tumor thickness, tumor progression, tumor metastasis, tumor distribution within the body, odor, molecular pathology, genomics, and/or tumor angiograms. In some embodiments, the one or more clinical data are used as clinical covariates and concatenated with the biomarker levels and input into a sparse rank regression model/algorithm (e.g., in order to identify, assess, and/or predict the aggressiveness or indolence of cancer (e.g., prostate cancer) in a subject). In one embodiment, the algorithm provides a cancer (e.g., prostate cancer) aggressiveness index value (e.g., 0, 1, 2, 3, or 4) that identifies and characterizes cancer in a subject (e.g., scaled such that a value of 0 characterizes the absence of cancer in the subject ranging to a value of 4 that characterizes the presence of highly aggressive cancer in the subject). In some embodiments, measuring a level of each of the biomarkers in the panel comprises measuring gene expression levels. The disclosure is not limited by how gene expression levels are measured. Indeed, any means of measuring gene expression levels may be used including, but not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpaII tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, or a combination thereof. In some embodiments, gene expression levels are measured by a sequencing technique such as, but not limited to, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing. MS-PET sequencing, mass spectrometry, and a combination thereof. In some embodiments, measuring a level of each of the biomarkers in the panel comprises measuring protein expression levels. The disclosure is not limited to any particular method of measuring protein expression levels. Exemplary methods of measuring protein expression levels include, but are not limited to, an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof. In some embodiments, measuring a level of each of the biomarkers in the panel comprises measuring by a qualitative assay, a quantitative assay, or a combination thereof. Exemplary quantitative assays include, but are not limited to, sequencing, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof. In some embodiments, the subject is a human.

In another aspect, the disclosure provides methods for detecting or diagnosing prostate cancer by using at least one or more (e.g., two, three, four, five, six, seven, eight, nine, ten, eleven, twelve, thirteen, fourteen, fifteen or more) markers selected from Table 1. Levels (e.g., gene expression levels, protein expression levels, or activity levels) of the selected markers may be measured from phagocytic cells (e.g., macrophages, monocytes, dendritic cells, and/or neutrophils) and from non-phagocytic cells (e.g., T cells), from a subject. Such levels then can be compared, e.g., the levels of the selected markers in the phagocytic cells and in the non-phagocytic cells to identify one or more differences between the measured levels, indicating whether the subject has prostate cancer. The identified difference(s) can also be used for assessing the risk of developing prostate cancer, prognosing prostate cancer, monitoring prostate cancer progression or regression, assessing the efficacy of a treatment for prostate cancer, or identifying a compound capable of ameliorating or treating prostate cancer.

In yet another aspect, the levels of the selected markers in the phagocytic cells may be compared to the levels of the selected markers in a control (e.g., a normal or healthy control subject, or a normal or healthy cell from the subject) to identify one or more differences between the measured levels, indicating whether the subject has prostate cancer, the prognosis of the cancer and the monitoring of the cancer. The identified difference(s) can also be used for assessing the risk of developing prostate cancer, prognosing prostate cancer, monitoring prostate cancer progression or regression, assessing the efficacy of a treatment for prostate cancer, or identifying a compound capable of ameliorating or treating prostate cancer.

In some embodiments, the disclosure provides a method for diagnosing or aiding in the diagnosis of prostate cancer in a subject, the method comprising the steps of:
a) measuring the levels of one or more (e.g., two, three, four, five, six, seven, eight, nine, ten, eleven, twelve, thirteen, fourteen, fifteen or more) markers selected from Table 1 in a population of the subject's macrophage or monocyte cells;
b) measuring the levels of the one or more selected markers in a population of the subject's non-phagocytic cells (e.g., T cells); and
c) identifying a difference between the measured levels of the one or more selected markers in steps a) and b), wherein the identified difference indicates that the subject has prostate cancer.

In some embodiments, the disclosure provides a method for diagnosing or aiding in the diagnosis of prostate cancer in a subject, the method comprising the steps of:
a) measuring the levels of one or more markers selected from CACNA1I, PDK4, TREML4, ODZ1, COL5A3, SLC11A1, GZMM, LOC283174, KRT72, SLC4A10, LBH, CHST15, COL6A2, ZNF683, ALPL, CSF1R, SCGB3A1, POM121L1P, SAMD3, FAM198B, NEBL, RASGRP1, KYNU, RTN1, CTSW, RORC, ABCB1, LMO2, S100A12, CD27, SH2D1A, PDZD4, CD300LB, FPR2, CLEC4F, AQP3, FOS, PTCH1, ASGR2, MARCKS, SIGLEC14, EGR1, MPEG1, SYNE2, POM121L4P, CD40LG, PYGL, MYCL1, CSMD1, BACH2, ENO2, LOXHD1, LILRA1, SLC44A5, CCDC64, IGSF9B, RORA, CD6, SCML4, RHOH, VCAN, CXCR3, PID1, EGR2, PRF1, NFAM1, SAPCD2, ADAMTSL4, TARP, PYHIN1, SKAP1, SPI1, MAL, KLRB1, KRT1, B3GAT1, ANPEP, CD86, MS4A14, FGFBP2, MS4A7, GPR15, CCR4, F13A1, CD247, EOMES, CD1D, C19orf59, LOC284837, TC2N, LCK, GZMB, GZMA, NAPSB, ZAP70, ALDH1A1, CD28, HIPIR, GPR56, S100A9, CLEC12A, GATA3, CD5, CST7, CLEC4E, TIGIT, HCK, ATP1A4, AQP9, GZMH, SULF2, LILRA3, CD14, IL7R, RIN2, VNN1, NCALD, TREM1, SYCP2L, and LGALS2 in a population of the subject's macrophage or monocyte cells;
b) measuring the levels of the one or more markers selected from CACNA1I, PDK4, TREML4, ODZ1, COL5A3, SLC11A1, GZMM, LOC283174, KRT72, SLC4A10, LBH, CHST15, COL6A2, ZNF683, ALPL, CSF1R, SCGB3A1, POM121L1P, SAMD3, FAM198B, NEBL, RASGRP1, KYNU, RTN1, CTSW, RORC, ABCB1, LMO2, S100A12, CD27, SH2D1A, PDZD4, CD300LB, FPR2, CLEC4F, AQP3, FOS, PTCH1, ASGR2, MARCKS, SIGLEC14, EGR1, MPEG1, SYNE2, POM121L4P, CD40LG, PYGL, MYCL1, CSMD1, BACH2, ENO2, LOXHD1, LILRA1, SLC44A5, CCDC64, IGSF9B, RORA, CD6, SCML4, RHOH, VCAN, CXCR3, PID1, EGR2, PRF1, NFAM1, SAPCD2, ADAMTSL4, TARP, PYHIN1, SKAP1, SPI1, MAL, KLRB1, KRT1, B3GAT1, ANPEP, CD86, MS4A14, FGFBP2, MS4A7, GPR15, CCR4, F13A1, CD247, EOMES, CD1D, C19orf59, LOC284837, TC2N, LCK, GZMB, GZMA, NAPSB, ZAP70, ALDH1A1, CD28, HIPIR, GPR56, S100A9, CLEC12A, GATA3, CD5, CST7, CLEC4E, TIGIT, HCK, ATP1A4, AQP9, GZMH, SULF2, LILRA3, CD14, IL7R, RIN2, VNN1, NCALD, TREM1, SYCP2L, and LGALS2 in a population of the subject's non-phagocytic cells (e.g., T cells) or from a cell free component from the subject; and
c) identifying a difference between the measured levels of the one or more selected markers in steps a) and b), wherein the identified difference indicates that the subject has prostate cancer.

In other embodiments, the disclosure provides a method for assessing the risk of developing prostate cancer in a subject, the method comprising the steps of:
a) measuring the levels of one or more (e.g., two, three, four, five, six, seven, eight, nine, ten, eleven, twelve, thirteen, fourteen, fifteen or more) markers selected from Table 1 in a population of the subject's macrophage or monocyte cells;
b) measuring the levels of the one or more selected markers in a population of the subject's non-phagocytic cells (e.g., T cells); and
c) identifying a difference between the measured levels of the one or more selected markers in steps a) and b), wherein the identified difference indicates that the subject has prostate cancer.

In other embodiments, the disclosure provides a method for assessing the risk of developing prostate cancer in a subject, the method comprising the steps of:
a) measuring the levels of one or more markers selected from CACNA1I, PDK4, TREML4, ODZ1, COL5A3, SLC11A1, GZMM, LOC283174, KRT72, SLC4A10, LBH, CHST15, COL6A2, ZNF683, ALPL, CSF1R, SCGB3A1, POM121L1P, SAMD3, FAM198B, NEBL, RASGRP1, KYNU, RTN1, CTSW, RORC, ABCB1, LMO2, S100A12, CD27, SH2D1A, PDZD4, CD300LB, FPR2, CLEC4F, AQP3, FOS, PTCH1, ASGR2, MARCKS, SIGLEC14, EGR1, MPEG1, SYNE2, POM121L4P, CD40LG, PYGL, MYCL1, CSMD1, BACH2, ENO2, LOXHD1, LILRA1, SLC44A5, CCDC64, IGSF9B, RORA, CD6, SCML4, RHOH, VCAN, CXCR3, PID1, EGR2, PRF1, NFAM1, SAPCD2, ADAMTSL4, TARP, PYHIN1, SKAP1, SPI1, MAL, KLRB1, KRT1, B3GAT1, ANPEP, CD86, MS4A14, FGFBP2, MS4A7, GPR15, CCR4, F13A1, CD247, EOMES, CD1D, C19orf59, LOC284837, TC2N, LCK, GZMB, GZMA, NAPSB, ZAP70, ALDH1A1, CD28, HIPIR, GPR56, S100A9, CLEC12A, GATA3, CD5, CST7, CLEC4E, TIGIT, HCK, ATP1A4, AQP9, GZMH, SULF2, LILRA3, CD14, IL7R, RIN2, VNN1, NCALD, TREM1, SYCP2L, and LGALS2 in a population of the subject's macrophage or monocyte cells;
b) measuring the levels of the one or more markers in a population of the subject's non-phagocytic cells (e.g., T cells) or from a cell free component from the subject; and
c) identifying a difference between the measured levels of the one or more selected markers in steps a) and b), wherein the identified difference indicates that the subject has said prostate cancer.

In some embodiments, the disclosure provides a method for prognosing or aiding in the prognosis of prostate cancer in a subject, the method comprising the steps of:
a) measuring the levels of one or more (e.g., two, three, four, five, six, seven, eight, nine, ten, eleven, twelve, thirteen, fourteen, fifteen or more) markers selected from Table 1 in a population of the subject's macrophage or monocyte cells;
b) measuring the levels of the one or more selected markers in a population of the subject's non-phagocytic cells (e.g., T cells); and
c) identifying a difference between the measured levels of the one or more selected markers in steps a) and b), wherein the identified difference indicates that the subject has prostate cancer.

In some embodiments, the disclosure provides a method for prognosing or aiding in the prognosis of prostate cancer in a subject, the method comprising the steps of:
a) measuring the levels of one or more markers selected from CACNA1I, PDK4, TREML4, ODZ1, COL5A3, SLC11A1, GZMM, LOC283174, KRT72, SLC4A10, LBH, CHST15, COL6A2, ZNF683, ALPL, CSF1R, SCGB3A1, POM121L1P, SAMD3, FAM198B, NEBL, RASGRP1, KYNU, RTN1, CTSW, RORC, ABCB1, LMO2, S100A12, CD27, SH2D1A, PDZD4, CD300LB, FPR2, CLEC4F, AQP3, FOS, PTCH1, ASGR2, MARCKS, SIGLEC14, EGR1, MPEG1, SYNE2, POM121L4P, CD40LG, PYGL, MYCL1, CSMD1, BACH2, ENO2, LOXHD1, LILRA1, SLC44A5, CCDC64, IGSF9B, RORA, CD6, SCML4, RHOH, VCAN, CXCR3, PID1, EGR2, PRF1, NFAM1, SAPCD2, ADAMTSL4, TARP, PYHIN1, SKAP1, SPI1, MAL, KLRB1, KRT1, B3GAT1, ANPEP, CD86, MS4A14, FGFBP2, MS4A7, GPR15, CCR4, F13A1, CD247, EOMES, CD1D, C19orf59, LOC284837, TC2N, LCK, GZMB, GZMA, NAPSB, ZAP70, ALDH1A1, CD28, HIPIR, GPR56, S100A9, CLEC12A, GATA3, CD5, CST7, CLEC4E, TIGIT, HCK, ATP1A4, AQP9, GZMH, SULF2, LILRA3, CD14, IL7R, RIN2, VNN1, NCALD, TREM1, SYCP2L, and LGALS2 in a population of the subject's macrophage or monocyte cells;
b) measuring the levels of the one or more markers in a population of the subject's non-phagocytic cells (e.g., T cells) or from a cell free component from the subject; and
c) identifying a difference between the measured levels of the one or more selected markers in steps a) and b), wherein the identified difference indicates that the subject has prostate cancer.

In some embodiments, the disclosure provides a method for assessing the efficacy of a treatment for prostate cancer in a subject comprising:
a) measuring the levels of one or more (e.g., two, three, four, five, six, seven, eight, nine, ten, eleven, twelve, thirteen, fourteen, fifteen or more) markers selected from Table 1 in a population of the subject's macrophage or monocyte cells before the treatment;
b) measuring the levels of the one or more selected markers in a population of the subject's non-phagocytic cells before the treatment;
c) identifying a first difference between the measured levels of the one or more selected markers in steps a) and b);
d) measuring the levels of the one or more selected markers in a population of the subject's macrophage or monocyte cells after the treatment;
e) measuring the levels of the one or more selected markers in a population of the subject's non-phagocytic cells after the treatment;
f) identifying a second difference between the measured levels of the one or more selected markers in steps d) and e); and
g) identifying a difference between the first difference and the second difference, wherein the difference identified in g) is indicative of the efficacy of the treatment for prostate cancer in the subject.

In some embodiments, the disclosure provides a method for assessing the efficacy of a treatment for prostate cancer in a subject comprising:
a) measuring the levels of one or more markers selected from Table 1 in a population of the subject's macrophage or monocyte cells before the treatment;
b) measuring the levels of the one or more markers in a population of the subject's non-phagocytic cells (e.g., T cells) or from a cell free component from the subject before the treatment;
c) identifying a first difference between the measured levels of the one or more selected markers in steps a) and b);
d) measuring the levels of the one or more selected markers in a population of the subject's macrophage or monocyte cells after the treatment;
e) measuring the levels of the one or more selected markers in a population of the subject's non-phagocytic cells (e.g., T cells) or from a cell free component from the subject after the treatment;
f) identifying a second difference between the measured levels of the one or more selected markers in steps d) and e); and
g) identifying a difference between the first difference and the second difference, wherein the difference identified in g) is indicative of the efficacy of the treatment for prostate cancer in the subject.

In other embodiments, the disclosure provides a method for monitoring the progression or regression of prostate cancer in a subject comprising:
a) measuring the levels of one or more (e.g., two, three, four, five, six, seven, eight, nine, ten, eleven, twelve, thirteen, fourteen, fifteen or more) markers selected from Table 1 in a population of the subject's macrophage or monocyte cells at a first time point;
b) measuring the levels of the one or more selected markers in a population of the subject's non-phagocytic cells at the first time point;
c) identifying a first difference between the measured levels of the one or more selected markers in steps a) and b);
d) measuring the levels of the one or more selected markers in a population of the subject's macrophage or monocyte cells at a second time point;
e) measuring the levels of the one or more selected markers in a population of the subject's non-phagocytic cells at the second time point;
f) identifying a second difference between the measured levels of the one or more selected markers in steps d) and e); and
g) identifying a difference between the first difference and the second difference, wherein the difference identified in g) is indicative of the progression or regression of said prostate cancer in the subject.

In other embodiments, the disclosure provides a method for monitoring the progression or regression of prostate cancer in a subject comprising:
a) measuring the levels of one or more markers selected from Table 1 in a population of the subject's macrophage or monocyte cells at a first time point;
b) measuring the levels of the one or more selected markers in a population of the subject's non-phagocytic cells (e.g., T cells) or from a cell free component from the subject at the first time point;
c) identifying a first difference between the measured levels of the one or more selected markers in steps a) and b);
d) measuring the levels of the one or more selected markers in a population of the subject's macrophage or monocyte cells at a second time point;
e) measuring the levels of the one or more selected markers in a population of the subject's non-phagocytic cells (e.g., T cells) or from a cell free component from the subject at the second time point;
f) identifying a second difference between the measured levels of the one or more selected markers in steps d) and e); and
g) identifying a difference between the first difference and the second difference, wherein the difference identified in g) is indicative of the progression or regression of prostate cancer in the subject.

In other embodiments, the disclosure provides a method for identifying a compound capable of ameliorating or treating prostate cancer in a subject comprising:
a) measuring the levels of one or more (e.g., two, three, four, five, six, seven, eight, nine, ten, eleven, twelve, thirteen, fourteen, fifteen or more) markers selected from Table 1 in a population of the subject's macrophage or monocyte cells before administering the compound to the subject;
b) measuring the levels of the one or more selected markers in a population of the subject's non-phagocytic cells (e.g., T cells) or from a cell free component from the subject before administering the compound to the subject;
c) identifying a first difference between the measured levels of the one or more selected markers in steps a) and b);
d) measuring the levels of the one or more selected markers in a population of the subject's macrophage or monocyte cells after the administration of the compound;
e) measuring the levels of the one or more selected markers in a population of the subject's non-phagocytic cells (e.g., T cells) or from a cell free component from the subject after the administration of the compound;
f) identifying a second difference between the measured levels of the one or more selected markers in steps d) and e); and
g) identifying a difference between the first difference and the second difference, wherein the difference identified in g) indicates that the compound is capable of ameliorating or treating prostate cancer in the subject.

In some embodiments, the selected markers are measured from the same population of non-phagocytic cells in steps b) or e). In some embodiments, the selected markers are measured from the different populations of non-phagocytic cells in steps b) or e). In some embodiments, at least two, three, four, five, six, seven, eight, nine, ten, eleven, twelve, thirteen, fourteen, fifteen or more markers are selected. The selected markers may be up-regulated or activated in the macrophage, monocyte, and/or neutrophil cells compared to the non-phagocytic cells (e.g., T cells) or from a cell free component from the subject, or, the selected markers may be down-regulated or inhibited in the macrophage, monocyte, and/or neutrophil cells compared to the non-phagocytic cells (e.g., T cells) or from a cell free component from the subject. In some embodiments, the methods comprise lysing the macrophage, monocyte, and/or neutrophil cells and the non-phagocytic cells before step a). In some embodiments, the methods comprise extracting the cellular contents from the macrophage, monocyte, and/or neutrophil cells and the non-phagocytic cells before step a). In some embodiments, the non-phagocytic cells are T cells, B cells, null cells, basophils, or mixtures thereof. In some embodiments, the macrophage, monocyte, and/or neutrophil cells are isolated from a bodily fluid sample, tissues, or cells of the subject. In other embodiments, the non-phagocytic cells are isolated from a bodily fluid sample, tissues, or cells of the subject. The disclosure is not limited by the type of bodily fluid sample. Indeed, multiple types of bodily fluid samples may be used including, but not limited to, blood, urine, stool, saliva, lymph fluid, cerebrospinal fluid, synovial fluid, cystic fluid, ascites, pleural effusion, fluid obtained from a pregnant woman in the first trimester, fluid obtained from a pregnant woman in the second trimester, fluid obtained from a pregnant woman in the third trimester, maternal blood, amniotic fluid, chorionic villus sample, fluid from a preimplantation embryo, maternal urine, maternal saliva, placental sample, fetal blood, lavage and cervical vaginal fluid, interstitial fluid, or ocular fluid. In some embodiments, the measured levels are gene expression levels. The disclosure is not limited by how the gene expression levels are measured. Indeed, any means of measuring gene expression levels described herein may be used. In some embodiments, the measured levels are protein expression levels. The present disclosure is also not limited by how protein expression levels are measured. A variety of non-limiting examples of how protein expression levels are measured are described herein. In some embodiments, the levels or activities are measured by a qualitative assay, a quantitative assay, or a combination thereof. Non-limiting examples of quantitative assays include sequencing, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing. MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof.

In some embodiments, the disclosure provides kits for measuring the levels of at least one or more (e.g., two, three, four, five, six, seven, eight, nine, ten, eleven, twelve, thirteen, fourteen, fifteen or more) markers from Table 1, comprising reagents for specifically measuring the levels of the one or more selected markers. The disclosure is not limited by how the markers are measured. In some embodiments, the reagents comprise one or more antibodies or fragments thereof, oligonucleotides, or aptamers.

In some embodiments, the disclosure provides kits for measuring the levels of at least one or more markers selected from CACNA1I, PDK4, TREML4, ODZ1, COL5A3, SLC11A1, GZMM, LOC283174, KRT72, SLC4A10, LBH, CHST15, COL6A2, ZNF683, ALPL, CSF1R, SCGB3A1, POM121L1P, SAMD3, FAM198B, NEBL, RASGRP1, KYNU, RTN1, CTSW, RORC. ABCB1, LMO2, S100A12, CD27, SH2D1A, PDZD4, CD300LB, FPR2, CLEC4F, AQP3, FOS, PTCH1, ASGR2, MARCKS, SIGLEC14, EGR1, MPEG1, SYNE2, POM121L4P, CD40LG, PYGL, MYCL1, CSMD1, BACH2, ENO2, LOXHD1, LILRA1, SLC44A5, CCDC64, IGSF9B, RORA, CD6, SCML4, RHOH, VCAN, CXCR3, PID1, EGR2, PRF1, NFAM1, SAPCD2, ADAMTSL4, TARP, PYHIN1, SKAP1, SPI1, MAL, KLRB1, KRT1, B3GAT1, ANPEP, CD86, MS4A14, FGFBP2, MS4A7, GPR15, CCR4, F13A1, CD247, EOMES, CD1D, C19orf59, LOC284837, TC2N. LCK, GZMB, GZMA, NAPSB, ZAP70, ALDH1A1, CD28, HIPIR, GPR56, S100A9, CLEC12A, GATA3, CD5, CST7, CLEC4E, TIGIT, HCK, ATP1A4, AQP9, GZMH, SULF2, LILRA3, CD14, IL7R, RIN2, VNN1, NCALD, TREM1, SYCP2L, and LGALS2, comprising reagents for specifically measuring the levels of the one or more selected markers. The disclosure is not limited by how the markers are measured. In some embodiments, the reagents comprise one or more antibodies or fragments thereof, oligonucleotides, or aptamers.

These and other embodiments of the subject disclosure will readily occur to those of skill in the art in view of the disclosure herein.

### DESCRIPTION OF THE DRAWINGS

FIG. 1 is a table showing cohort demographics for discovery and validation data sets. p-values are for significance testing for differences between the discovery and validation sets. Missing represents the percentage of cases where data was missing or unavailable.

FIG. 2 shows ROC analysis results for various models showing discovery and validation AUC values with confidence intervals. Results are shown for discovery set (disc), and independent validation set (val). Clinical variables used in models and validation AUC results by age tertial are shown.

FIG. 3 shows ROC curves for genomics only CD2, CD14, and CD14/CD2 ratio models. AUC values and confidence intervals are shown in FIG. 2.

FIG. 4 shows ROC curves for CD14/CD2 ratio model compared to those models including age, PSA, and PSAD. AUC values and confidence intervals are shown in FIG. 2.

FIG. 5 shows top-ranked, enriched pathways and ontologies represented by the 120 genes in the best performing model according to MSigDB hallmark (A), KEGG (B), and gene ontology biological processes (C). Only terms that had a false discovery rate <0.1 (or <0.01 for gene ontology (C)) are shown.

FIG. 6 provides data integrity information for the models.

FIG. 7 provides marker signatures obtained via the disclosed methods.

FIG. 8 provides significance tests of the models.

FIG. 9 provides UMAP for XPR+age+PSA.

### DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +/−20% or +/−10%, more preferably +/−5%, even more preferably +/−1%, and still more preferably +/−0.1% from the specified value, as such variations are appropriate to perform the disclosed methods.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the terms “biomarker” or “marker” or “genomic marker” or “genomic covariate” or “biological marker” refer to an analyte (e.g., a nucleic acid, DNA, RNA, peptide, protein, or metabolite) that can be objectively measured and evaluated as an indicator for a biological process. In some embodiments, a marker is differentially detectable in phagocytes and is indicative of the presence or absence of prostate cancer. An analyte is differentially detectable if it can be distinguished quantitatively or qualitatively in phagocytes compared to a control, e.g., a normal or healthy control or non-phagocytic cells.

The terms “sample” or “biological sample” or “component” as used herein, refers to a sample of biological fluid, tissue, or cells, in a healthy and/or pathological state obtained from a subject. Such samples include, but are not limited to, blood, bronchial lavage fluid, sputum, saliva, urine, amniotic fluid, lymph fluid, tissue or fine needle biopsy samples, peritoneal fluid, cerebrospinal fluid, nipple aspirates, and includes supernatant from cell lysates, lysed cells, cellular extracts, and nuclear extracts.

The terms “patient,” “subject,” “individual,” and the like are used interchangeably herein and refer to either a human or a non-human animal. These terms include mammals, such as humans, primates, livestock animals (e.g., bovines, porcines), companion animals (e.g., canines, felines) and rodents (e.g., mice and rats).

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors for developing cancer. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the type and/or stage of cancer is not known. The term further includes people who previously had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, etc.

As used herein, the term “subject at risk for cancer” refers to a subject with one or more risk factors for developing a specific cancer. Risk factors include, but are not limited to, gender, age, genetic predisposition, environmental exposure, and previous incidents of cancer, preexisting non-cancer diseases, and lifestyle.

As used herein, the term “characterizing cancer in a subject” refers to the identification of one or more properties of a cancer sample in a subject, including but not limited to, the presence of benign, pre-cancerous or cancerous tissue and the stage of the cancer. In one non-limiting example, compositions and methods of the disclosure are utilized to characterize cancer in a subject (e.g., to identify the aggressiveness or indolence of prostate cancer) in a subject.

As used herein, the terms “normal control”, “healthy control”, and “not-diseased cells” likewise mean a sample (e.g., cells, serum, tissue) taken from a source (e.g., subject, control subject, cell line) that does not have the condition or disease being assayed and therefore may be used to determine the baseline for the condition or disorder being measured. A control subject refers to any individual that has not been diagnosed as having the disease or condition being assayed. It is also understood that the control subject, normal control, and healthy control, include data obtained and used as a standard, i.e. it can be used over and over again for multiple different subjects. In other words, for example, when comparing a subject sample to a control sample, the data from the control sample could have been obtained in a different set of experiments, for example, it could be an average obtained from a number of healthy subjects and not actually obtained at the time the data for the subject was obtained.

The term “diagnosis” as used herein refers to methods by which the skilled artisan can estimate and/or determine whether or not a patient is suffering from a given disease or condition. In some embodiments, the term “diagnosis” also refers to staging (e.g., Stage I, II, III, or IV) of cancer. The skilled artisan often makes a diagnosis on the basis of one or more diagnostic indicators, e.g., a marker, the presence, absence, amount, or change in amount of which is indicative of the presence, severity, or absence of the condition.

The term “prognosis” as used herein refers to is used herein to refer to the likelihood of prostate cancer progression, including recurrence of prostate cancer.

The terms “comprise(s).” “include(s),” “having.” “has,” “can.” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising.” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The “area under curve” or “AUC” refers to area under a ROC curve. AUC under a ROC curve is a measure of accuracy. An AUC of 1 represents a perfect test, whereas an AUC of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, at least approximately 0.995, at least approximately 0.990, at least approximately 0.850, at least approximately 0.800, at least approximately 0.750, at least approximately 0.700, at least approximately 0.650, or at least approximately 0.600.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

A “receiver operating characteristic” curve or “ROC” curve refers to a graphical plot that illustrates the performance of a binary classifier system as its discrimination threshold is varied. For example, an ROC curve can be a plot of the true positive rate against the false positive rate for the different possible cutoff points of a diagnostic test. It is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate. The ROC curve demonstrates the tradeoff between sensitivity and specificity (any increase in sensitivity will be accompanied by a decrease in specificity); the closer the curve follows the left-hand border and then the top border of the ROC space, the more accurate the test; the closer the curve comes to the 45-degree diagonal of the ROC space, the less accurate the test; the slope of the tangent line at a cutoff point gives the likelihood ratio (LR) for that value of the test; and the area under the curve is a measure of test accuracy.

A variety of cell types, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, red blood cells, platelets, interstitial fluid, cerebral spinal fluid, etc. Cell types and tissues may also include lymph fluid, cerebrospinal fluid, a fluid collected by A tissue or cell type may be provided by removing a sample of cells from a human and a non-human animal, but can also be accomplished by using previously isolated cells (e.g., isolated by another person, at another time, and/or for another purpose). Archival tissues, such as those having treatment or outcome history, may also be used.

“Sensitivity” of an assay as used herein refers to the proportion of subjects for whom the outcome is positive that are correctly identified as positive.

“Specificity” of an assay as used herein refers to the proportion of subjects for whom the outcome is negative that are correctly identified as negative.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistical significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

As used herein, “treating” prostate cancer refers to taking steps to obtain beneficial or desired results, including clinical results. Beneficial or desired clinical results include, but are not limited to, alleviation or amelioration of one or more symptoms associated with diseases or conditions.

As used herein, “administering” or “administration of” a compound or an agent to a subject can be carried out using one of a variety of methods known to those skilled in the art. For example, a compound or an agent can be administered, intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. In some embodiments, a compound or an agent is administered orally, e.g., to a subject by ingestion, or intravenously, e.g., to a subject by injection. In some embodiments, the orally administered compound or agent is in an extended release or slow release formulation or administered using a device for such slow or extended release.

### DETAILED DESCRIPTION

The present disclosure relates to compositions and methods for assessing prostate cancer (e.g., identification of the aggressiveness or indolence of prostate cancer) in a subject. The compositions and methods include obtaining subject specific information (e.g., age, digital rectal exam (DRE) data, prostate volume or density, total prostate-specific antigen (PSA)) and obtaining a biological sample from a subject and determining a measurement for a panel of biomarkers in the biological sample.

The disclosure provides methods for identifying, assessing and/or predicting the aggressiveness or indolence of cancer (e.g., prostate cancer) in a subject (e.g., a subject suspected of having cancer, a subject diagnosed with a cancer, or a subject at risk for cancer). In some embodiments, the disclosure provides a method for identifying, assessing and/or predicting the aggressiveness or indolence of prostate cancer (e.g., in a patient previously diagnosed with prostate cancer).

Genomic expressions present in all cells within an individual are affected by and change consequent to a variety of factors. These factor include, but are not limited to, intrinsic inter-individual (e.g., gender, ethnic background, etc.) variations; age-related (temporal) variations; extracellular “milieu” stimuli (e.g., recent food/drink intake, recent vaccination, exposure to infectious organisms, etc.); the presence of one or more specific diseases (e.g., cancer, that a blood test aims to detect via detection of an immunological response); and other disease/conditions unrelated to the disease that conventional tests aim to detect. Each of these factors lead to an orchestrated upregulation and downregulation and silencing of certain genes.

Accordingly, in conventional blood-based disease assays (e.g., cancer assays, rheumatoid arthritis, infectious disease), a diseased patient's profile (e.g., from plasma, PBMCs, a WBC subpopulation, etc.) is compared to that of an individual identified not to have the disease (a control subject or panel of subjects) with the hope/expectation of identifying a disease signature. However, since the baseline/background signatures of the individual with the “Disease” are specific to his/her genomic profile and that of the “Control” are specific to his/her genomic profile, such intrinsic inter-individual differences have, and will always, impede the identification of a valid disease signature.

The disclosure provides assays utilizing Subtraction Normalized Expressions of Phagocytes (SNEP) to identify biomarkers (e.g., a nucleic acid, DNA, RNA, peptide, protein, or metabolite) that alone, or in combination with patient clinical information, find utility in the identification of a disease signature (e.g., that is used for detecting and/or identifying disease in a subject). In SNEP, intrinsic signatures not related to the disease are filtered out and the “normalized data” from the patient and the control are used to identify a disease specific signature. Thus, in some embodiments, the disclosure provides one or more disease signatures (e.g., one or more prostate cancer disease signatures) and methods of using the signature(s) to identify, assess, and/or predict various facets of disease in a subject. For example, in some embodiments, detecting or identifying disease in a subject comprises identifying, assessing and/or predicting the aggressiveness or indolence of cancer (e.g., prostate cancer) in a subject (e.g., in a patient previously diagnosed with prostate cancer) using one or more of the signatures described herein.

In other aspects, the disclosure provides use of non-normal continuous clinical covariates, namely total prostate specific antigen (PSA), prostate volume, and PSA density (PSAD) (e.g., that are log-transformed before analyses). In some aspects, models are built using a two-step procedure using unsupervised variance-based transcript down-selection and classification by gradient boosting tree-based model (LightGBM) (See, e.g., [43]). These models can then be used to make predictions and/or determinations on the validation set. In some aspects, models are developed on the discovery set for each cell type alone (CD2 & CD14) and/or for the ratio (CD14/CD2)—these models are considered the genomic expression only models. The performance of various clinical data (Age, Race, DRE, Family History, PSA and PSAD) can be evaluated and then combined with the ratio of CD14/CD2 to investigate and determine possible enhancements to model performance.

Model performance can be evaluated by determining the area under the curve (AUC) of the receiver operating characteristic (ROC) curve [44]. Differences in performance were assessed using the DeLong test [45]. All analyses were performed in R, including the gene ontology and pathway associations using the enrichR package [46-48] (See Example 1).

As described in Example 1, over one thousand patients were used to identify signatures of disease. CD2⁺ T cells and CD14⁺ monocytes and/or macrophages were isolated from patients, RNA extracted and whole genome, RNA sequencing performed (23,368 transcripts (gene symbols), corresponding to 29.8±7.5 million and 33.9±7.5 million mapped reads for CD2 and CD14 samples were obtained). Gene expression levels were used to develop predictive models that correlate to adverse pathologic features. The immune-transcriptomic model with the highest performance for predicting adverse pathology, based on a subtraction of the log-transformed expression signals of the two cell types, displayed an area under the curve (AUC) of the receiver operating characteristic of 0.70. The addition of biomarkers in combination with traditional clinical risk factors (age, serum prostate-specific antigen (PSA), PSA density, race, digital rectal examination, and family history) enhanced the AUC to 0.91 and 0.83 for the training and validation sets, respectively. The disclosure provides markers (identified by this approach) that identify and represent specific pathway associations relevant to (prostate) cancer biology. The disclosure further provides markers that represent increased phagocytic activity in conjunction with cancer-associated (mis-) regulation. In further aspects, the disclosure also provides differential gene expression of circulating immune cells that provides insight into the cellular immune response to early tumor development and immune surveillance.

As disclosed herein, the immune-transcriptomic profiling of purified populations of phagocytic cells (e.g., CD14+ monocytes) and non-phagocytic cells (e.g., CD2+ lymphocytes) provides a unique look into the pathways that are up or down regulated in patients with aggressive prostate cancer (e.g., as defined by adverse pathologic features compared to biopsy negative controls and men with indolent pathologic disease). The specific genes in these pathways can be used to identify, prognose and/or predict adverse prostate cancer pathology. The present disclosure not only provides an understanding of the underlying biological phenomena and the cell types involved in immunologic responses to cancer, but also into potential novel biomarker strategies to identify, prognose, predict, treat, and/or manage cancer.

The present disclosure provides information regarding two distinct immunologic responses to prostate cancer (e.g., early-stage prostate cancer), the phagocytic and the immune response mechanisms, each harboring specific involvement in their response to an ongoing oncogenic process. Exploring this response as a ratio between these two cell type populations provides more information than exploring an individual cell type alone. An advantage of the methods of the disclosure is the normalization effect (noise reduction) that using the ratio has on setting the baseline overall activity state of the immune system and showing the upregulation of multiple pathways including phagocytic and cancer pathways.

The disclosure provides gene sets and associated pathways uncovered by examining differential gene expression of circulating immune cells in the setting of early-stage prostate cancer that highlight two different response mechanisms to early tumor development: a) the tumor phagocytosis and b) immune response mechanisms (See Example 1). In some aspects, the genes associated with clinically significant cancer provides identification of novel immune modulation therapeutic targets as well as markers for the development of prognostic and diagnostic models.

In some aspects, the clinico-immuno-genomic blood cell based approach utilizing gradient boosting described herein demonstrates that (i) concurrent CD14+/CD2+ sequencing from the same patient is beneficial to (a) filter out genomic signatures not associated with the disease, (b) achieve strong concordance with tissue biopsy testing results, and/or (c) substantially enhance the AUCs obtained from various current PCa clinical risk factors only, and/or (ii) the differential transcriptomic profiles of CD14+ and CD2+ cell populations are associated with and can predict adverse pathologic features of clinically localized prostate cancer. Compositions and methods disclosed herein find use in the identification, prognosis, treatment and/or management of prostate cancer patients.

In one non-limiting example, the disclosure provides an Aggressiveness Index according to utilized clinical parameters based on: maximum Gleason grade, maximal cross section surface area of a core, and number of positive cores to generate an aggressiveness index scored between 0-4, where a Score of 0 meant no evidence of cancer on 12 core or more biopsy; a Score of 1 meant low grade⁺ and low volume⁺ (i.e., Grade 1, 1-2 cores up to 10%; or Grade 2, 1-2 cores up to 5%); a Score of 2 meant low grade⁺⁺ and low volume⁺⁺ (i.e., Grade 1, 3-5 cores [20-40%]; or Grade 2, 3-4 cores [10-20%]; or Grade 3, 1-2 cores [1-5%]); a Score of 3 meant intermediate grade and intermediate volume (i.e., Grade 1, 6-12 cores [50-100%]; or Grade 2, 5-9 cores [30-70%]; or Grade 3, 3-6 cores [10-50%]; or Grade 4, 1-2 cores [1-5%]; or Grade 5, 1 core [1-2%]); and a Score of 4 meant high grade and high volume (i.e., Grade 2-3, >5 cores [>50%]; or Grade 4, >2 cores [>10%]; or Grade 5, >1 core [>1%]).

Throughout experiments conducted during development of embodiments of the disclosure (e.g., described in the Example), a subset of inputs (biomarker and clinical covariates) were identified by the model as predictive, termed “PC signature”, that were solely responsible for the predictions made by the model, inputs not in the signature (with zero model coefficients), were ignored. After training the model on 713 patients, 120 markers/genomic covariates were identified (See Table 1).

TABLE 1
120 genomic covariates identified from best performing model.

| Gene/marker | Feature | Gain | Cover | Frequency |
|---|---|---|---|---|
| CACNA1I | 2108 | 0.025766 | 0.035051 | 0.04119 |
| PDK4 | 10863 | 0.017544 | 0.031015 | 0.025172 |
| TREML4 | 15410 | 0.01672 | 0.029177 | 0.029748 |
| ODZ1 | 10457 | 0.015323 | 0.020517 | 0.022883 |
| COL5A3 | 3125 | 0.014935 | 0.013994 | 0.016018 |
| SLC11A1 | 13337 | 0.014919 | 0.012909 | 0.016018 |
| GZMM | 5961 | 0.012267 | 0.016889 | 0.016018 |
| LOC283174 | 8022 | 0.011337 | 0.022752 | 0.016018 |
| KRT72 | 7266 | 0.011234 | 0.016873 | 0.016018 |
| SLC4A10 | 13561 | 0.01004 | 0.025582 | 0.029748 |
| LBH | 7339 | 0.008867 | 0.011891 | 0.01373 |
| CHST15 | 2875 | 0.00853 | 0.013168 | 0.01373 |
| COL6A2 | 3127 | 0.008276 | 0.017677 | 0.018307 |
| ZNF683 | 16950 | 0.008198 | 0.012375 | 0.018307 |
| ALPL | 471 | 0.007993 | 0.012865 | 0.016018 |
| CSF1R | 3313 | 0.00765 | 0.011995 | 0.016018 |
| SCGB3A1 | 12932 | 0.006532 | 0.013207 | 0.011442 |
| POM121L1P | 11401 | 0.006352 | 0.013163 | 0.01373 |
| SAMD3 | 12838 | 0.005979 | 0.015596 | 0.01373 |
| FAM198B | 4727 | 0.005812 | 0.005791 | 0.006865 |
| NEBL | 10013 | 0.005515 | 0.01384 | 0.011442 |
| RASGRP1 | 12144 | 0.005334 | 0.009149 | 0.006865 |
| KYNU | 7284 | 0.005151 | 0.012964 | 0.01373 |
| RTN1 | 12763 | 0.005114 | 0.018954 | 0.016018 |
| CTSW | 3398 | 0.004649 | 0.006958 | 0.006865 |
| RORC | 12545 | 0.004534 | 0.006523 | 0.009153 |
| ABCB1 | 35 | 0.0043 | 0.008423 | 0.009153 |
| LMO2 | 7559 | 0.004273 | 0.008714 | 0.006865 |
| S100A12 | 12803 | 0.004083 | 0.00785 | 0.004577 |
| CD27 | 2499 | 0.00407 | 0.002659 | 0.004577 |
| SH2D1A | 13195 | 0.004 | 0.010933 | 0.011442 |
| PDZD4 | 10886 | 0.003794 | 0.011263 | 0.009153 |
| CD300LB | 2508 | 0.003776 | 0.003787 | 0.006865 |
| FPR2 | 5247 | 0.003741 | 0.005263 | 0.004577 |
| CLEC4F | 2970 | 0.003716 | 0.005296 | 0.006865 |
| AQP3 | 716 | 0.003545 | 0.006601 | 0.006865 |
| FOS | 5211 | 0.003435 | 0.003617 | 0.006865 |
| PTCH1 | 11819 | 0.003297 | 0.008759 | 0.009153 |
| ASGR2 | 917 | 0.003279 | 0.005885 | 0.004577 |
| MARCKS | 8736 | 0.003197 | 0.004354 | 0.004577 |
| SIGLEC14 | 13270 | 0.003108 | 0.003942 | 0.006865 |
| EGR1 | 4209 | 0.003056 | 0.00959 | 0.011442 |
| MPEG1 | 9468 | 0.002912 | 0.003303 | 0.004577 |
| SYNE2 | 14488 | 0.002882 | 0.004944 | 0.004577 |
| POM121L4P | 11402 | 0.002781 | 0.001073 | 0.002288 |
| CD40LG | 2524 | 0.002733 | 0.002587 | 0.002288 |
| PYGL | 11944 | 0.002557 | 0.003364 | 0.004577 |
| MYCL1 | 9748 | 0.002481 | 0.002587 | 0.002288 |
| CSMD1 | 3320 | 0.002458 | 0.00093 | 0.002288 |
| BACH2 | 1148 | 0.002422 | 0.002004 | 0.004577 |
| ENO2 | 4354 | 0.002408 | 0.004421 | 0.004577 |
| LOXHD1 | 8389 | 0.002393 | 0.002224 | 0.002288 |
| LILRA1 | 7426 | 0.002157 | 0.004459 | 0.006865 |
| SLC44A5 | 13550 | 0.002156 | 0.002598 | 0.004577 |
| CCDC64 | 2360 | 0.002108 | 0.003837 | 0.006865 |
| IGSF9B | 6563 | 0.002051 | 0.001795 | 0.002288 |
| RORA | 12543 | 0.00205 | 0.001607 | 0.002288 |
| CD6 | 2536 | 0.002025 | 0.006832 | 0.009153 |
| SCML4 | 12940 | 0.001998 | 0.002642 | 0.002288 |
| RHOH | 12375 | 0.001963 | 0.0054 | 0.006865 |
| VCAN | 16018 | 0.001944 | 0.000661 | 0.002288 |
| CXCR3 | 3443 | 0.001938 | 0.011775 | 0.006865 |
| PID1 | 11055 | 0.001899 | 0.009215 | 0.006865 |
| EGR2 | 4210 | 0.001772 | 0.005637 | 0.006865 |
| PRF1 | 11607 | 0.001745 | 0.003143 | 0.004577 |
| NFAM1 | 10062 | 0.001724 | 0.008841 | 0.006865 |
| SAPCD2 | 12854 | 0.001711 | 0.004316 | 0.004577 |
| ADAMTSL4 | 228 | 0.001689 | 0.003705 | 0.006865 |
| TARP | 14583 | 0.001621 | 0.003898 | 0.006865 |
| PYHIN1 | 11947 | 0.001613 | 0.000947 | 0.002288 |
| SKAP1 | 13310 | 0.001563 | 0.004756 | 0.004577 |
| SPI1 | 14129 | 0.001539 | 0.001409 | 0.002288 |
| MAL | 8630 | 0.001512 | 0.002274 | 0.002288 |
| KLRB1 | 7225 | 0.001476 | 0.0024 | 0.002288 |
| KRT1 | 7257 | 0.001434 | 0.002791 | 0.006865 |
| B3GAT1 | 1118 | 0.001431 | 0.003336 | 0.006865 |
| ANPEP | 613 | 0.001378 | 0.003925 | 0.002288 |
| CD86 | 2551 | 0.001241 | 0.001206 | 0.002288 |
| MS4A14 | 9597 | 0.001223 | 0.008169 | 0.006865 |
| FGFBP2 | 5039 | 0.001218 | 0.003017 | 0.002288 |
| MS4A7 | 9602 | 0.001214 | 0.006204 | 0.004577 |
| GPR15 | 5754 | 0.001089 | 0.00332 | 0.006865 |
| CCR4 | 2447 | 0.001 | 0.001431 | 0.002288 |
| F13A1 | 4554 | 0.000966 | 0.003925 | 0.002288 |
| CD247 | 2497 | 0.000932 | 0.00262 | 0.002288 |
| EOMES | 4377 | 0.000926 | 0.0018 | 0.002288 |
| CD1D | 2485 | 0.000907 | 0.004652 | 0.006865 |
| C19orf59 | 1677 | 0.000884 | 0.001861 | 0.002288 |
| LOC284837 | 8060 | 0.000868 | 0.002703 | 0.002288 |
| TC2N | 14676 | 0.000828 | 0.002422 | 0.002288 |
| LCK | 7345 | 0.000793 | 0.007046 | 0.006865 |
| GZMB | 5958 | 0.000734 | 0.00076 | 0.002288 |
| GZMA | 5957 | 0.000731 | 0.004299 | 0.004577 |
| NAPSB | 9872 | 0.000678 | 0.003187 | 0.004577 |
| ZAP70 | 16363 | 0.000651 | 0.002648 | 0.004577 |
| ALDH1A1 | 421 | 0.000618 | 0.001261 | 0.002288 |
| CD28 | 2502 | 0.000597 | 0.001811 | 0.002288 |
| HIP1R | 6154 | 0.000567 | 0.000875 | 0.002288 |
| GPR56 | 5783 | 0.000528 | 0.003837 | 0.002288 |
| S100A9 | 12811 | 0.000492 | 0.000451 | 0.002288 |
| CLEC12A | 2953 | 0.000483 | 0.004619 | 0.004577 |
| GATA3 | 5405 | 0.000441 | 0.001261 | 0.002288 |
| CD5 | 2529 | 0.000415 | 0.001332 | 0.002288 |
| CST7 | 3342 | 0.000377 | 0.000347 | 0.002288 |
| CLEC4E | 2969 | 0.000337 | 0.002114 | 0.002288 |
| TIGIT | 14883 | 0.000279 | 0.003187 | 0.004577 |
| HCK | 6039 | 0.000266 | 0.002653 | 0.002288 |
| ATP1A4 | 1003 | 0.000243 | 0.002521 | 0.002288 |
| AQP9 | 720 | 0.000234 | 0.00104 | 0.002288 |
| GZMH | 5959 | 0.000206 | 0.00093 | 0.002288 |
| SULF2 | 14420 | 0.000147 | 0.002059 | 0.002288 |
| LILRA3 | 7428 | 0.000139 | 0.000974 | 0.002288 |
| CD14 | 2472 | 9.39E−05 | 0.000407 | 0.002288 |
| IL7R | 6627 | 7.09E−05 | 0.000363 | 0.002288 |
| RIN2 | 12401 | 4.08E−05 | 0.002951 | 0.002288 |
| VNN1 | 16051 | 3.37E−05 | 0.001156 | 0.004577 |
| NCALD | 9908 | 2.78E−05 | 0.000886 | 0.002288 |
| TREM1 | 15406 | 2.75E−05 | 0.000479 | 0.002288 |
| SYCP2L | 14476 | 7.65E−06 | 0.001718 | 0.002288 |
| LGALS2 | 7399 | 5.01E−06 | 0.0006 | 0.002288 |

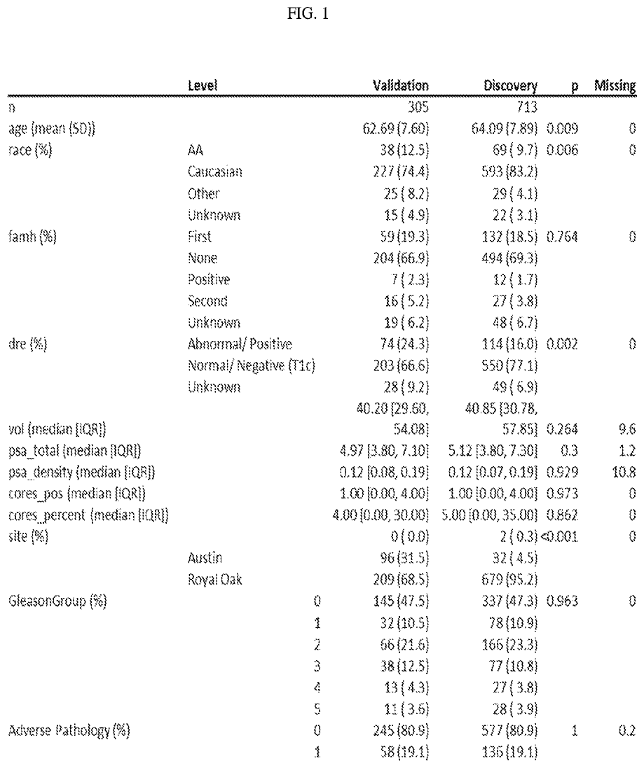

PC signatures identified contained multiple inputs, for example, clinical covariates including age, DRE, prostate volume and/or density, and total PSA, as well as biomarker covariates (See Table 1 and FIG. 1).

Thus, in some aspects, the disclosure utilizes compositions and methods described herein and/or one or more signatures identified herein to stratify cancer patients. For example, in one embodiment, the disclosure provides assays and/or one or more signatures identified herein to stratify patients with indolent prostate disease from those with aggressive prostate cancer (e.g., that require life-saving treatments). Accordingly, in some embodiments, compositions and methods described herein find use in clinical assessment and management of subjects (e.g., patients at risk for cancer (e.g., prostate cancer)). For example, in some embodiments, assays and/or one or more signatures identified herein classify a patient as definitive for treatment (e.g., with one or more anti-cancer therapies) or as needing only surveillance (e.g., no treatment).

In some embodiments, compositions and methods of the disclosure (e.g., assays and/or one or more signatures identified herein) provide a clinician the ability to stratify a patient into either a treatment group (e.g., requiring cancer treatment and/or therapies) or a surveillance group (e.g., not requiring immediate treatment) without need for a physically invasive biopsy. That is, in some embodiments, compositions and methods of the disclosure are used to avoid unnecessary patient biopsies (e.g., prostate cancer biopsy), repeat biopsies, and/or the pain and suffering and risk factors/side effects consequent to biopsies (e.g., in men under active surveillance for prostate cancer). In some embodiments, compositions and methods of the disclosure benefit men diagnosed with prostate cancer in that the compositions and methods (assays and/or one or more signatures identified herein) identify patients needing further workup and/or treatment.

In one embodiment, the present disclosure provides biological markers and methods of using them to detect a cancer (e.g., prostate cancer). The present disclosure is based on the discovery that one or more markers described in Table 1 are useful in diagnosing prostate cancer, either alone, or when assessed in the context of one or more clinical covariates (e.g., age, digital rectal exam (DRE) data, prostate volume and/or density, total prostate-specific antigen (PSA)). In one embodiment, the disclosure provides a cancer (e.g., prostate cancer) aggressiveness index value (e.g., 0, 1, 2, 3, or 4) that identifies and characterizes cancer in a subject (e.g., scaled such that a value of 0 characterizes the absence of cancer in the subject ranging to a value of 4 that characterizes the presence of highly aggressive cancer in the subject). In some embodiments, one or more clinical covariates are concatenated with one or more biomarker levels and input into a sparse rank regression model in order to generate a prostate cancer aggressiveness index.

For example, in some embodiments, by measuring the levels of the biomarkers (e.g., gene expression levels, protein expression levels, or protein activity levels) in a population of phagocytes (e.g., macrophages, monocytes, or neutrophils) from a human subject, one can provide a reliable diagnosis for prostate cancer (e.g., identifying, assessing and/or predicting the aggressiveness or indolence of prostate cancer).

As used herein, a “level” of a marker of this disclosure can be qualitative (e.g., presence or absence) or quantitative (e.g., amounts, copy numbers, or dosages). In some embodiments, a level of a marker at a zero value can indicate the absence of this marker. The levels of any marker of this disclosure can be measured in various forms. For example, the level can be a gene expression level, a RNA transcript level, a protein expression level, a protein activity level, and/or an enzymatic activity level.

The markers of this disclosure can be used in methods for diagnosing or aiding in the diagnosis of prostate cancer by comparing levels (e.g., gene expression levels, or protein expression levels, or protein activities) of one or more prostate cancer markers (e.g., nucleic acids or proteins) between phagocytes (e.g., macrophages, monocytes, or neutrophils) and non-phagocytic cells (e.g., T cells) or a cell free component taken from the same individual. This disclosure also provides methods for assessing the risk of developing prostate cancer, prognosing the cancer, monitoring the cancer progression or regression, assessing the efficacy of a treatment, or identifying a compound capable of ameliorating or treating the cancer.

Compositions and methods of the disclosure find use in the identification, characterization, and classification (e.g., via one or more assays and/or methods disclosed) of cancer in a subject. In particular embodiments, the compositions and methods of the disclosure are applied to prostate cancer. As used herein, “prostate cancer” means any cancer of the prostate including, but not limited to, adenocarcinoma and small cell carcinoma.

In some embodiments, the disclosure provides a method for identifying, assessing and/or predicting the aggressiveness or indolence of cancer (e.g., prostate cancer) in a subject (e.g., a subject suspected of having cancer, a subject diagnosed with a cancer, or a subject at risk for cancer). In some embodiments, the disclosure provides a method for identifying, assessing and/or predicting the aggressiveness or indolence of prostate cancer (e.g., in a patient previously diagnosed with prostate cancer).

In some embodiments, the disclosure provides a method of measuring a panel of biomarkers in a subject comprising obtaining a biological sample from the subject; determining a measurement for the panel of biomarkers in the biological sample, wherein the panel of biomarkers comprise one or more (e.g., two, three, four, five, six, seven, eight, nine, ten, eleven, twelve, thirteen, fourteen, fifteen or more) biomarkers of Table 1 and wherein the measurement comprises measuring a level of each of the biomarkers in the panel. In some embodiments, measuring the panel of biomarkers in the subject identifies, assesses, and/or predicts the aggressiveness or indolence of cancer (e.g., prostate cancer) in a subject (e.g., a subject suspected of having cancer, a subject diagnosed with a cancer, or a subject at risk for cancer). In some embodiments, the biological sample comprises CD2⁺ cells and/or CD14⁺ cells. In one embodiment, determining a measurement for the panel of biomarkers in the biological sample comprises measuring a level of each of the biomarkers in the panel in CD2⁺ cells and/or CD14⁺ cells. In one embodiment, the method further comprises obtaining one or more clinical data from the subject selected from the group consisting of age, race, digital rectal exam (DRE), prostate volume and/or density, and total prostate-specific antigen (PSA). In some embodiments, the one or more clinical data are used as clinical covariates and concatenated with the biomarker levels and input into a sparse rank regression model (e.g., in order to identify, assess, and/or predict the aggressiveness or indolence of cancer (e.g., prostate cancer) in a subject). In some embodiments, measuring a level of each of the biomarkers in the panel comprises measuring gene expression levels.

In some embodiments, the disclosure provides a method of measuring a panel of biomarkers in a subject comprising obtaining a biological sample from the subject; determining a measurement for the panel of biomarkers in the biological sample, wherein the panel of biomarkers comprise one or more (e.g., two, three, four, five, six, seven, eight, nine, ten, eleven, twelve, thirteen, fourteen, or fifteen) biomarkers from CACNA1I, PDK4, TREML4, ODZ1, COL5A3, SLC11A1, GZMM, LOC283174, KRT72, SLC4A10, LBH, CHST15, COL6A2, ZNF683, ALPL, CSF1R, SCGB3A1, POM121L1P, SAMD3, FAM198B, NEBL, RASGRP1, KYNU, RTN1, CTSW, RORC, ABCB1, LMO2, S100A12, CD27, SH2D1A, PDZD4, CD300LB, FPR2, CLEC4F. AQP3, FOS, PTCH1, ASGR2, MARCKS, SIGLEC14, EGR1, MPEG1, SYNE2, POM121L4P, CD40LG, PYGL, MYCL1, CSMD1, BACH2, ENO2, LOXHD1, LILRA1, SLC44A5, CCDC64, IGSF9B, RORA, CD6, SCML4, RHOH, VCAN, CXCR3, PID1, EGR2, PRF1, NFAM1, SAPCD2, ADAMTSL4, TARP, PYHIN1, SKAP1, SPI1, MAL, KLRB1, KRT1, B3GAT1, ANPEP, CD86, MS4A14, FGFBP2, MS4A7, GPR15, CCR4, F13A1, CD247, EOMES, CD1D, C19orf59, LOC284837, TC2N, LCK, GZMB, GZMA, NAPSB, ZAP70, ALDH1A1, CD28, HIPIR, GPR56, S100A9, CLEC12A, GATA3, CD5, CST7, CLEC4E, TIGIT, HCK, ATP1A4, AQP9, GZMH, SULF2, LILRA3, CD14, IL7R, RIN2, VNN1, NCALD, TREM1, SYCP2L, and LGALS2, and wherein the measurement comprises measuring a level of each of the biomarkers in the panel. In some embodiments, measuring the panel of biomarkers in the subject identifies, assesses, and/or predicts the aggressiveness or indolence of cancer (e.g., prostate cancer) in a subject (e.g., a subject suspected of having cancer, a subject diagnosed with a cancer, or a subject at risk for cancer). In some embodiments, the biological sample comprises CD2⁺ cells and/or CD14⁺ cells. In some embodiments, determining a measurement for the panel of biomarkers in the biological sample comprises measuring a level of each of the biomarkers in the panel in CD2⁺ cells and/or CD14⁺ cells. In one embodiment, the method further comprises obtaining one or more clinical data from the subject selected from the group consisting of age, race, digital rectal exam (DRE), prostate volume and/or density, and total prostate-specific antigen (PSA). In some embodiments, the one or more clinical data are used as clinical covariates and concatenated with the biomarker levels and input into a sparse rank regression model (e.g., in order to identify, assess, and/or predict the aggressiveness or indolence of cancer (e.g., prostate cancer) in a subject). In some embodiments, measuring a level of each of the biomarkers in the panel comprises measuring gene expression levels.

The disclosure is not limited by how gene expression levels are measured. Indeed, any means of measuring gene expression levels may be used including, but not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpaII tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, or a combination thereof. In some embodiments, gene expression levels are measured by a sequencing technique such as, but not limited to, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing, MS-PET sequencing, mass spectrometry, and a combination thereof. In some embodiments, measuring a level of each of the biomarkers in the panel comprises measuring protein expression levels.

The disclosure is not limited to any particular method of measuring protein expression levels. Exemplary methods of measuring protein expression levels include, but are not limited to, an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof. In some embodiments, measuring a level of each of the biomarkers in the panel comprises measuring by a qualitative assay, a quantitative assay, or a combination thereof. Exemplary quantitative assays include, but are not limited to, sequencing, direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof. In some embodiments, the subject is a human.

The disclosure also provides a kit for performing measurement at least two (e.g., two, three, four, five, six, seven, eight, nine, ten, eleven, twelve or more) of the markers listed in Table 1, wherein the kit comprises reagents for measuring the at least two markers.

In another aspect, the methods (e.g., diagnosis of prostate cancer, prognosis of prostate cancer, or assessing the risk of developing prostate cancer) provided in the disclosure comprise: a) measuring the levels of one or more (e.g., two, three, four, five, six, seven, eight, nine, ten, eleven, twelve, thirteen, fourteen, fifteen or more) markers from Table 1 in a population of a subject's macrophage or monocyte cells; b) measuring the levels of one or more of the selected markers in a population of a subject's non-phagocytic cells (e.g., T-cells, B-cells, null cells, basophils or the mixtures of two more non-phagocytic cells); comparing the measured levels in step a) to the measured levels in step b) and further identifying a difference between the measured levels of a) and b). In some aspects, the identified difference is indicative of the diagnosis (e.g., presence or absence), prognosis (e.g., lethal outcome, or tumor stage), or the risk of developing prostate cancer.

In another aspect, the methods (e.g., diagnosis of prostate cancer, prognosis of prostate cancer, or assessing the risk of developing prostate cancer) provided in the disclosure comprise: a) measuring the levels of one or more (e.g., two, three, four, five, six, seven, cight, nine, ten, eleven, twelve, thirteen, fourteen, fifteen or more) markers from Table 1 in a population of a subject's macrophage or monocyte cells; and b) identifying a difference between the measured levels of the selected markers in step a) and the levels of the selected markers in a control (e.g., a healthy control cell, or a control cell from a healthy subject). The identified difference is indicative of the diagnosis (e.g., presence or absence), prognosis (e.g., lethal outcome, or tumor stage), or the risk of developing prostate cancer. In some embodiments, the selected markers are up-regulated in prostate cancer patients. In some embodiments, the selected markers are down-regulated in prostate cancer patients. In some embodiments, the selected markers comprise at least one marker that is up-regulated and at least one marker that is down-regulated. In some embodiments, the method of diagnosing, prognosing, and/or assessing the aggressiveness and/or indolence of prostate cancer provided by the disclosure (e.g., via measuring the levels of one or more markers from Table 1 optionally in combination with one or more clinical covariates) provides a better diagnostic, prognostic and/or assessment than a Gleason score of the prostate cancer, and/provides a diagnostic, prognostic and/or assessment of prostate cancer not heretofore available.

In another aspect, the disclosure provides methods for assessing the efficacy of a treatment for prostate cancer, monitoring the progression or regression of prostate cancer, or identifying a compound capable of ameliorating or treating prostate cancer, respectively, in a subject comprising: a) measuring the levels of one or more (e.g., two, three, four, five, six, seven, eight, nine, ten, eleven, twelve, thirteen, fourteen, fifteen or more) markers from Table 1 in a population of the subject's macrophage or monocyte cells before the treatment, or at a first time point, or before administration of the compound, respectively; b) measuring the levels of the one or more selected markers in a population of the subject's non-phagocytic cells before the treatment, or at the first time point, or before administration of the compound, respectively; c) identifying a first difference between the measured levels of the one or more selected markers in steps a) and b); d) measuring the levels of the one or more selected markers in a population of the subject's macrophage or monocyte cells after the treatment, or at a second time point, or after administration of the compound, respectively; c) measuring the levels of the one or more selected markers in a population of the subject's non-phagocytic cells after the treatment, or at the second time point, or after administration of the compound, respectively; f) identifying a second difference between the measured levels of the one or more selected markers in steps d) and c); and g) identifying a difference between the first difference and the second difference, wherein the difference identified in g) is indicative of the efficacy of the treatment for the prostate cancer, or the progression or regression of the prostate cancer, or whether the compound is capable of ameliorating or treating the prostate cancer, respectively, in the subject.

In another aspect, the disclosure provides methods for assessing the efficacy of a treatment for prostate cancer, monitoring the progression or regression of prostate cancer, or identifying a compound capable of ameliorating or treating prostate cancer, respectively, in a subject comprising: a) measuring the levels of one or more (e.g., two, three, four, five, six, seven, eight, nine, ten, eleven, twelve, thirteen, fourteen, fifteen or more) markers from Table 1 in a population of the subject's macrophage or monocyte cells before the treatment, or at a first time point, or before administration of the compound, respectively; b) identifying a first difference between the measured levels of the one or more selected markers in step (a) and the levels of the one or more selected markers in a control (e.g., a healthy control cell, or a control cell from a healthy subject) before the treatment, or at the first time point, or before administration of the compound, respectively; c) measuring the levels of the one or more selected markers in a population of the subject's macrophage or monocyte cells after the treatment, or at a second time point, or after administration of the compound, respectively; d) identifying a second difference between the measured levels of the one or more selected markers in step c) and the levels of the one or more selected markers in a control after the treatment, or at the second time point, or after administration of the compound, respectively; and e) identifying a difference between the first difference and the second difference, wherein the difference identified in c) is indicative of the efficacy of the treatment for the prostate cancer, or the progression or regression of the prostate cancer, or whether the compound is capable of ameliorating or treating the prostate cancer, respectively, in the subject.

In some embodiments, two sub-populations of phagocytic cells (e.g., monocytes) are used in the methods of this disclosure, i.e., phagocytic cells that have a DNA content greater than 2n (the >2n phagocytic cells) and phagocytic cells that have a DNA content of 2n (the =2n phagocytic cells). In such embodiments, the levels of the selected markers in the >2n phagocytic cells are compared to the =2n phagocytic cells to identify one or more differences. The identified differences indicate whether the subject has prostate cancer, or has a risk of developing prostate cancer, or has a progressing or progressive prostate cancer.

In some embodiments, the levels of two, three, four, five, six, seven, eight, nine, ten, eleven, twelve, thirteen, fourteen, fifteen, or more of the markers from Table 1 are measured. In some embodiments, the levels of one or more (e.g., two, three, four, five, six, seven, eight, nine, ten, eleven, twelve, thirteen, fourteen, fifteen or more) markers from Table 1 are measured, and are concatenated with one or more clinical data (clinical covariates) and input into a sparse rank regression model/algorithm in order to identify, assess and/or predict the aggressiveness or indolence of cancer (e.g., prostate cancer) in a subject (e.g., a subject suspected of having cancer, a subject diagnosed with a cancer, or a subject at risk for cancer). The disclosure is not limited by the type of clinical data utilized. Indeed, a variety of clinical data may be used including, but not limited to, age, race, digital rectal exam (DRE), prostate volume and/or density, total prostate-specific antigen (PSA), tumor stage, tumor grade, tumor size, tumor visual characteristics, tumor growth, tumor thickness, tumor progression, tumor metastasis, tumor distribution within the body, odor, molecular pathology, genomics, and/or tumor angiograms.

In some embodiments, at least one or more of the selected markers from Table 1 may be substituted with a biological marker different from any of the selected markers. In some embodiments, such biological markers may be known markers for prostate cancer. In some embodiments, such biological markers and the substituted selected markers may belong to the same signaling or biological pathway (e.g., tumor phagocytosis pathway, immune response pathway, a protein synthesis pathway, Th1 cytokine production pathway, transcription pathway, programmed cell death pathway), or may have similar biological function or activity (e.g., protein synthesis, Th1 cytokine production, nucleotide binding, protein binding, transcription, a receptor for purines coupled to G-proteins, inhibition of programmed cell death, neutrophil activation, an IL-8 receptor, an HSP70-interacting protein, stimulating ATPase activity), or may be regulated by a common protein, or may belong to the same protein complex (e.g., an HSP70 protein complex).

In some embodiments, a population of a subject's macrophage, monocyte, and/or neutrophil cells is used as the selected phagocytic cells for measuring the levels of the selected markers and a population of the subject's T-cells is used as the selected non-phagocytic cells for measuring the levels of the selected markers. In other embodiments, a population of the subject's neutrophil cells is used as the selected phagocytic cells for measuring the levels of the selected markers and a population of the subject's T-cells is used as the selected non-phagocytic cells for measuring the levels of the selected markers.

The gene names/descriptions provided in Table 1 are merely illustrative. The markers of this disclosure encompass all forms and variants of any specifically described/identified markers, including, but not limited to, polymorphic or allelic variants, isoforms, mutants, derivatives, precursors including nucleic acids and pro-proteins, cleavage products, and structures comprised of any of the markers as constituent subunits of the fully assembled structure.

Each embodiment described herein may be combined with any other embodiment described herein.

Methods using the prostate cancer markers described herein provide high specificity. sensitivity, and accuracy in detecting and diagnosing prostate cancer. The methods also eliminate the “inequality of baseline” that is known to occur among individuals due to intrinsic (e.g., age, gender, ethnic background, health status and the like) and temporal variations in marker expression. Additionally, by using a comparison of phagocytes and non-phagocytes from the same individual, and/or the statistical methods disclosed herein, the methods also allow detection, diagnosis, and treatment to be personalized to the individual. Accordingly, in some embodiments, the disclosure provides non-invasive assays (e.g., liquid biopsy) for the detection (e.g., early detection) of prostate cancer, i.e., before the prostate cancer can be diagnosed by conventional diagnostic techniques, e.g., imaging techniques, and, therefore, provide a foundation for improved decision-making relative to the needs and strategies for intervention, prevention, and treatment of individuals with such disease or condition.

The methods described herein are supported by whole genome microarray data of total RNA samples isolated from phagocytic cells (e.g., macrophages, monocytes, dendritic cells, and/or neutrophils) and from non-phagocytic cells (e.g., T cells). The samples were obtained from human subjects with and without prostate cancer. The data demonstrate that macrophage/monocyte-T cell comparisons easily and accurately differentiate between prostate cancer patients and human subjects without prostate cancer.

The methods of this disclosure can be used together with any known diagnostic methods, such as physical inspection, visual inspection, biopsy, scanning, histology, radiology, imaging, ultrasound, use of a commercial kit, genetic testing, immunological testing, analysis of bodily fluids, or monitoring neural activity.

Phagocytic cells that can be used in the methods of this disclosure include all types of cells that are capable of ingesting various types of substances (e.g., apoptotic cells, infectious agents, dead cells, viable cells, cell-free DNAs, cell-free RNAs, cell-free proteins). In some embodiments, the phagocytic cells are neutrophils, macrophages, monocytes, dendritic cells, foam cells, mast cells, cosinophils, or keratinocytes. In some embodiments, the phagocytic cells can be a mixture of different types of phagocytic cells. In some embodiments, the phagocytic cells can be activated phagocytic cells, e.g., activated macrophages, monocytes, or neutrophils. In some embodiments, a phagocyte is a histiocyte, e.g., a Langerhans cell.

In certain embodiments, markers used in the methods of disclosure are up-regulated or activated in phagocytes (e.g., macrophages, monocytes, or neutrophils) compared to non-phagocytes. In certain embodiments, markers used in the methods of disclosure are down-regulated or inhibited in phagocytes (e.g., macrophages, monocytes, or neutrophils) compared to non-phagocytes. As used herein, “up-regulation or up-regulated” can refer to an increase in expression levels (e.g., gene expression or protein expression), gene copy numbers, gene dosages, and other qualitative or quantitative detectable state of the markers. Similarly, “down-regulation or down-regulated” can refer to a decrease in expression levels, gene copy numbers, gene dosages, and other qualitative or quantitative detectable state of the markers. As used herein, “activation or activated” can refer to an active state of the marker, e.g., a phosphorylation state, a DNA methylation state, or a DNA acetylation state. Similarly, “inhibition or inhibited” can refer to a repressed state or an inactivated state of the marker, e.g., a de-phosphorylation state, a ubiquitination state, or a DNA de-methylation state.

In certain embodiments, methods of this disclosure also comprise at least one of the following steps before determination of various levels: i) lysing the phagocytic or non-phagocytic cells; and ii) extracting cellular contents from the lysed cells. Any known cell lysis and extraction methods can be used herein. In certain embodiments, at least one or more prostate cancer markers are present in the phagocytes. In certain embodiments, there is no marker present in the cellular contents of the non-phagocytic cells.

In certain embodiments, the phagocytic cells and/or non-phagocytic cells are isolated from a bodily fluid sample, tissues, or population of cells. Exemplary bodily fluid samples can be whole blood, urine, stool, saliva, lymph fluid, cerebrospinal fluid, synovial fluid, cystic fluid, ascites, pleural effusion, fluid obtained from a pregnant woman in the first trimester, fluid obtained from a pregnant woman in the second trimester, fluid obtained from a pregnant woman in the third trimester, maternal blood, amniotic fluid, chorionic villus sample, fluid from a preimplantation embryo, maternal urine, maternal saliva, placental sample, fetal blood, lavage and cervical vaginal fluid, interstitial fluid, buccal swab sample, sputum, bronchial lavage, Pap smear sample, or ocular fluid. In some embodiments, the phagocytic cells or non-phagocytic cells are isolated from white blood cells.

In the methods of this disclosure, cell separation/isolation/purification methods are used to isolate populations of cells from bodily fluid sample, cells, or tissues of a subject. A skilled worker can use any known cell separation/isolation/purification techniques to isolate phagocytic cells and non-phagocytic cells from a bodily fluid. Exemplary techniques include, but are not limited to, using antibodies, flow cytometry, fluorescence activated cell sorting, filtration, gradient-based centrifugation, elution, microfluidics, immunomagnetic separation technique, multiple size immuno-beads filtration techniques, fluorescent-magnetic separation technique, nanostructure, quantum dots, high throughput microscope-based platform, or a combination thereof.

In certain embodiments, the phagocytic cells and/or non-phagocytic cells are isolated by using a product secreted by the cells. In certain embodiments, the phagocytic cells and/or non-phagocytic cells are isolated by using a cell surface target (e.g., receptor protein) on the surface of the cells. In some embodiments, the cell surface target is a protein that has been engulfed by phagocytic cells. In some embodiments, the cell surface target is expressed by cells on their plasma membranes. In some embodiments, the cell surface target is an exogenous protein that is translocated on the plasma membranes, but not expressed by the cells (e.g., the phagocytic cells). In some embodiments, the cell surface target is a marker of prostate cancer.

In certain aspects of the methods described herein, analytes include nucleic acids, proteins, or any combinations thereof. In certain aspects of the methods described herein, markers include nucleic acids, proteins, or any combinations thereof. As used herein, the term “nucleic acid” is intended to include DNA molecules (e.g., cDNA or genomic DNA), RNA molecules (e.g., mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNAs), microRNA (miRNAs), small nucleolar RNA (snoRNAs), ribosomal RNA (rRNA), transfer RNA (tRNA), small interfering RNA (siRNA), heterogeneous nuclear RNAs (hnRNA), or small hairpin RNA (shRNA). In some embodiments, the nucleic acid is a transrenal nucleic acid. A transrenal nucleic acid is an extracellular nucleic acid that is excreted in the urine. Sec, e.g., U.S. Patent Publication No. 20100068711 and U.S. Patent Publication No. 20120021404.

As used herein, the term “amino acid” includes organic compounds containing both a basic amino group and an acidic carboxyl group. Included within this term are natural amino acids (e.g., L-amino acids), modified and unusual amino acids (e.g., D-amino acids and.beta.-amino acids), as well as amino acids which are known to occur biologically in free or combined form but usually do not occur in proteins. Natural protein occurring amino acids include alanine, arginine, asparagine, aspartic acid, cysteine, glutamic acid, glutamine, glycine, histidine, isoleucine, leucine, lysine, methionine, phenylalanine, serine, threonine, tyrosine, tryptophan, proline, and valine. Natural non-protein amino acids include arginosuccinic acid, citrulline, cysteine sulfuric acid, 3,4-dihydroxyphenylalanine, homocysteine, homoserine, ornithine, 3-monoiodotyrosine, 3,5-diiodotryosine, 3,5,5-triiodothyronine, and 3,3′,5,5′-tetraiodothyronine. Modified or unusual amino acids include D-amino acids, hydroxylysine, 4-hydroxyproline, N-Cbz-protected amino acids, 2.4-diaminobutyric acid, homoarginine, norleucine, N-methylaminobutyric acid, naphthylalanine, phenylglycine,.alpha.-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

As used herein, the term “peptide” includes compounds that consist of two or more amino acids that are linked by means of a peptide bond. Peptides may have a molecular weight of less than 10,000 Daltons, less than 5,000 Daltons, or less than 2,500 Daltons. The term “peptide” also includes compounds containing both peptide and non-peptide components, such as pseudopeptide or peptidomimetic residues or other non-amino acid components. Such compounds containing both peptide and non-peptide components may also be referred to as a “peptide analog.”

As used herein, the term “protein” includes compounds that consist of amino acids arranged in a linear chain and joined together by peptide bonds between the carboxyl and amino groups of adjacent amino acid residues. Proteins used in methods of the disclosure include, but are not limited to, amino acids, peptides, antibodies, antibody fragments, cytokines, lipoproteins, or glycoproteins.

As used herein, the term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, and antibody fragments (e.g., Fab or F (ab′).sub.2, and Fv). For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, Conn., 1994, page 71 and Chapter 6.

As used herein, the term “cytokine” refers to a secreted protein or active fragment or mutant thereof that modulates the activity of cells of the immune system. Examples of cytokines include, without limitation, interleukins, interferons, chemokines, tumor necrosis factors, colony-stimulating factors for immune cell precursors, and the like.

As used herein, the term “lipoprotein” includes negatively charged compositions that comprise a core of hydrophobic cholesteryl esters and triglyceride surrounded by a surface layer of amphipathic phospholipids with which free cholesterol and apolipoproteins are associated. Lipoproteins may be characterized by their density (e.g. very-low-density lipoprotein (VLDL), low-density lipoprotein (LDL) and high density lipoprotein (HDL)), which is determined by their size, the relative amounts of lipid and protein. Lipoproteins may also be characterized by the presence or absence of particular modifications (e.g. oxidization, acetylation, or glycation).

As used herein, the term “glycoprotein” includes glycosides which have one or more oligo- or polysaccharides covalently attached to a peptide or protein. Exemplary glycoproteins can include, without limitation, immunoglobulins, members of the major histocompatibility complex, collagens, mucins, glycoprotein IIb/IIIa, glycoprotein-41 (gp41) and glycoprotein-120 (gp12), follicle-stimulating hormone, alpha-fetoprotein, erythropoietin, transferrins, alkaline phosphatase, and lectins.

In some embodiments of the disclosure, a sample may comprise one or more stabilizers for a cell or an analyte such as DNA, RNA, and/or protein. For example, a sample may comprise a DNA stabilizer, an RNA stabilizer, and/or a protein stabilizer. Stabilizers are well known in the art and include, for example, DNAse inhibitors, RNAse inhibitors, and protease inhibitors or equivalents thereof.

In some embodiments of the disclosure, levels of at least one or more prostate cancer markers are compared. This comparison can be quantitative or qualitative. Quantitative measurements can be taken using any of the assays described herein. For example, sequencing, direct sequencing, random shotgun sequencing, Sanger dideoxy termination sequencing, targeted sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing. SOLiD™ sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof.

Quantitative comparisons can include statistical analyses such as t-test, ANOVA, Krustal-Wallis, Wilcoxon, Mann-Whitney, odds ratio, or any statistical method described herein (e.g., in Example 1). Quantitative differences can include differences in the levels of markers between levels or differences in the numbers of markers present between levels, and combinations thereof. Examples of levels of the markers can be, without limitation, gene expression levels, nucleic acid levels, and protein levels. Qualitative differences can include, but are not limited to, activation and inactivation, protein degradation, nucleic acid degradation, and covalent modifications.

In certain embodiments of the disclosure, the level is a nucleic acid level or a protein level, or a combination thereof. The level can be qualitatively or quantitatively determined.

A nucleic acid level can be, without limitation, a genotypic level, a single nucleotide polymorphism level, a gene mutation level, a gene copy number level, a DNA methylation level, a DNA acetylation level, a chromosome dosage level, a gene expression level, or a combination thereof.

The nucleic acid level can be determined by any methods known in the art to detect genotypes, single nucleotide polymorphisms, gene mutations, gene copy numbers, DNA methylation states, DNA acetylation states, chromosome dosages. Exemplary methods include, but are not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpalI tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, co-amplification at lower denaturation temperature-PCR (COLD-PCR), multiplex PCR, methyl-binding PCR analysis, or a combination thereof.

As used herein, the term “sequencing” is used in a broad sense and refers to any technique known in the art that allows the order of at least some consecutive nucleotides in at least part of a nucleic acid to be identified, including without limitation at least part of an extension product or a vector insert. Exemplary sequencing techniques include targeted sequencing, single molecule real-time sequencing, whole transcriptome shotgun sequencing (“RNA-seq”), electron microscopy-based sequencing, transistor-mediated sequencing, direct sequencing, random shotgun sequencing, Sanger dideoxy termination sequencing, exon sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing. Solexa Genome Analyzer sequencing, SOLID™ sequencing, MS-PET sequencing, mass spectrometry, and a combination thereof. In some embodiments, sequencing comprises an detecting the sequencing product using an instrument, for example but not limited to an ABI PRISM™ 377 DNA Sequencer, an ABI PRISM™ 310, 3100, 3100-Avant, 3730, or 3730xI Genetic Analyzer, an ABI PRISM™ 3700 DNA Analyzer, or an Applied Biosystems SOLID™ System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel signature sequencing (MPSS).

In further embodiments of the disclosure, a protein level can be a protein expression level, a protein activation level, or a combination thereof. In some embodiments, a protein activation level can comprise determining a phosphorylation state, an ubiquitination state, a myristylation state, or a conformational state of the protein.

A protein level can be detected by any methods known in the art for detecting protein expression levels, protein phosphorylation state, protein ubiquitination state, protein myristylation state, or protein conformational state. In some embodiments, a protein level can be determined by an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof.

As used herein, the “difference” between different levels detected by the methods of this disclosure can refer to different gene copy numbers, different DNA, RNA, or protein expression levels, different DNA methylation states, different DNA acetylation states, and different protein modification states. The difference can be a difference greater than 1 fold (e.g., 1.0 to 100.0 fold, or greater). In some embodiments, the difference is a 1.05-fold, 1.1-fold, 1.2-fold, 1.3-fold, 1.4-fold, 1.5-fold, 2-fold, 2.5-fold, 3-fold, 4-fold, 5-fold, 6-fold, 7-fold, 8-fold, 9-fold, or 10-fold difference. In some embodiments, the difference is any fold difference between 1-10, 2-10, 5-10, 10-20, or 10-100 fold.

In some embodiments, the difference is differential gene expression (DGE), e.g. DGE of phagocytes vs. non-phagocytes. DGE can be measured as X=log₂ (Y_{P})−log₂ (Y_{NP}). The DGE may be any number, provided that it is significantly different between the phagocytes and the non-phagocytes. For example, a 2-fold increase in gene expression could be represented as X=log₂ (Y_{P})−log₂ (Y_{NP})=log₂ (Y_{P/Y2NP})=log₂ (2)=1, while a 2-fold decrease in gene expression could be represented as X=log₂ (Y_{P})−log₂ (Y_{NP})=log₂ (Y_{2P/YNP})=log₂ (1/2)=−1. Down-regulated genes have X<0, while up-regulated genes have X>0. See, e.g., Efron, J Am Stat Assoc 104:1015-1028 (2009).

A general principle of assays to detect markers involves preparing a sample or reaction mixture that may contain the marker (e.g., one or more of DNA, RNA, or protein) and a probe under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxysuccinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In certain exemplary embodiments, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, U.S. Pat. Nos. 5,631,169 and 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C, 1991, Anal. Chem. 63:2338 2345 and Szabo et al, 1995, Curr. Opin. Struct. Biol. 5:699 705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas and Minton (1993) Trends Biochem. Sci. 18:284). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard (1998) J. Mol. Recognit. 11:141; Hage and Tweed (1997) J. Chromatogr. B. Biomed. Sci. Appl. 12:499). Gel electrophoresis may also be employed to separate complexed assay components from unbound components (sec. e.g., Ausubel et al, ed., Current Protocols in Molecular Biology, John Wiley & Sons, New York, 1987 1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In certain exemplary embodiments, the level of mRNA corresponding to the marker can be determined either by in situ and/or by in vitro formats in a biological sample using methods known in the art. Many expression detection methods use isolated RNA. For in vitro methods, any RNA isolation technique that does not select against the isolation of mRNA can be utilized for the purification of RNA from blood cells (see, e.g., Ausubel et al, ed., Current Protocols in Molecular Biology, John Wiley & Sons, New York 1987 1999). Additionally, large numbers of cells and/or samples can readily be processed using techniques well known to those of skill in the art, such as, for example, the single-step RNA isolation process of Chomczynski (1989, U.S. Pat. No. 4,843,155).

Isolated mRNA can be used in hybridization or amplification assays that include, but are not limited to, Southern or Northern analyses, polymerase chain reaction analyses and probe arrays. In certain exemplary embodiments, a diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a marker of the present disclosure. Other suitable probes for use in the diagnostic assays of the disclosure are described herein. Hybridization of an mRNA with the probe indicates that the marker in question is being expressed.

In one format, the mRNA is immobilized on a solid surface and contacted with a probe. for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array. A skilled artisan can readily adapt known mRNA detection methods for use in detecting the level of mRNA encoded by the markers of the present disclosure.

An alternative method for determining the level of mRNA corresponding to a marker of the present disclosure in a sample involves the process of nucleic acid amplification, e.g., by RT-PCR (the experimental embodiment set forth in U.S. Pat. Nos. 4,683,195 and 4,683,202), COLD-PCR (Li et al. (2008) Nat. Med. 14:579), ligase chain reaction (Barany, 1991, Proc. Natl. Acad. Sci. USA, 88:189), self-sustained sequence replication (Guatelli et al., 1990, Proc. Natl. Acad. Sci. USA 87:1874), transcriptional amplification system (Kwoh et al. (1989) Proc. Natl. Acad. Sci. USA 86:1173), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the sample (e.g., a bodily fluid (e.g., blood cells)) prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in a patient sample from one source to a patient sample from another source, e.g., to compare a population of phagocytic from an individual to a population of non-phagocytic cells from the individual.

In one embodiment of this disclosure, a protein or polypeptide corresponding to a marker is detected. In certain embodiments, an agent for detecting a protein or polypeptide can be an antibody capable of binding to the polypeptide, such as an antibody with a detectable label. As used herein, the term “labeled.” with regard to a probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. Antibodies can be polyclonal or monoclonal. An intact antibody, or a fragment thereof (e.g., Fab or F (ab′) 2) can be used. In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well known supports or carriers include glass, polystyrene, polypropylene, polyethylene, dextran, nylon, amylases, natural and modified celluloses, polyacrylamides, gabbros, magnetite and the like.

A variety of formats can be employed to determine whether a sample contains a protein that binds to a given antibody. Examples of such formats include, but are not limited to, competitive and non-competitive immunoassay, enzyme immunoassay (EIA), radioimmunoassay (RIA), antigen capture assays, two-antibody sandwich assays, Western blot analysis, enzyme linked immunosorbant assay (ELISA), a planar array, a colorimetric assay, a chemiluminescent assay, a fluorescent assay, and the like. Immunoassays, including radioimmmunoassays and enzyme-linked immunoassays, are useful in the methods of the present disclosure. A skilled artisan can readily adapt known protein/antibody detection methods for use in determining whether cells (e.g., bodily fluid cells such as blood cells) express a marker of the present disclosure.

One skilled in the art will know many other suitable carriers for binding antibody or antigen, and will be able to adapt such support for use with the present disclosure. For example, protein isolated from cells (e.g., bodily fluid cells such as blood cells) can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

In certain exemplary embodiments, assays are provided for diagnosis, prognosis, assessing the risk of developing prostate cancer, assessing the efficacy of a treatment, monitoring the progression or regression of prostate cancer, and identifying a compound capable of ameliorating or treating prostate cancer. An exemplary method for these methods involves obtaining a bodily fluid sample from a test subject, isolating phagocytes and non-phagocytes, and contacting the phagocytes and non-phagocytes with a compound or an agent capable of detecting one or more of the markers of the disease or condition, e.g., marker nucleic acid (e.g., mRNA, genomic DNA), marker peptide (e.g., polypeptide or protein), marker lipid (e.g., cholesterol), or marker metabolite (e.g., creatinine) such that the presence of the marker is detected. In one embodiment, an agent for detecting marker mRNA or genomic DNA is a labeled nucleic acid probe capable of hybridizing to marker mRNA or genomic DNA. The nucleic acid probe can be, for example, a full-length marker nucleic acid or a portion thereof. Other suitable probes for use in the diagnostic assays of the disclosure are described herein.

As used herein, a compound capable of ameliorating or treating prostate cancer can include, without limitations, any substance that can improve symptoms or prognosis, prevent progression of the prostate cancer, promote regression of the prostate cancer, or eliminate the prostate cancer.

The methods of the disclosure can also be used to detect genetic alterations in a marker gene, thereby determining if a subject with the altered gene is at risk for developing prostate cancer characterized by misregulation in a marker protein activity or nucleic acid expression. In certain embodiments, the methods include detecting, in phagocytes, the presence or absence of a genetic alteration characterized by an alteration affecting the integrity of a gene encoding a marker peptide and/or a marker gene. For example, such genetic alterations can be detected by ascertaining the existence of at least one of: 1) a deletion of one or more nucleotides from one or more marker genes; 2) an addition of one or more nucleotides to one or more marker genes; 3) a substitution of one or more nucleotides of one or more marker genes, 4) a chromosomal rearrangement of one or more marker genes; 5) an alteration in the level of a messenger RNA transcript of one or more marker genes; 6) aberrant modification of one or more marker genes, such as of the methylation pattern of the genomic DNA; 7) the presence of a non-wild type splicing pattern of a messenger RNA transcript of one or more marker genes; 8) a non-wild type level of a one or more marker proteins; 9) allelic loss of one or more marker genes; and 10) inappropriate post-translational modification of one or more marker proteins. As described herein, there are a large number of assays known in the art which can be used for detecting alterations in one or more marker genes.

In certain embodiments, detection of the alteration involves the use of a probe/primer in a polymerase chain reaction (PCR) (see, e.g., U.S. Pat. Nos. 4,683,195, 4,683,202 and 5,854,033), such as real-time PCR, COLD-PCR (Li et al. (2008) Nat. Med. 14:579), anchor PCR, recursive PCR or RACE PCR, or, alternatively, in a ligation chain reaction (LCR) (see, e.g., Landegran et al. (1988) Science 241:1077; Prodromou and Pearl (1992) Protein Eng. 5:827; and Nakazawa et al. (1994) Proc. Natl. Acad. Sci. USA 91:360), the latter of which can be particularly useful for detecting point mutations in a marker gene (see Abravaya et al. (1995) Nucleic Acids Res. 23:675). This method can include the steps of collecting a sample of cell free bodily fluid from a subject, isolating nucleic acid (e.g., genomic, mRNA or both) from the sample, contacting the nucleic acid sample with one or more primers which specifically hybridize to a marker gene under conditions such that hybridization and amplification of the marker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli et al., (1990) Proc. Natl. Acad. Sci. USA 87:1874), transcriptional amplification system (Kwoh et al., (1989) Proc. Natl. Acad. Sci. USA 86:1173), Q Beta Replicase (Lizardi et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in one or more marker genes from a sample can be identified by alterations in restriction enzyme cleavage patterns. For example, sample and control DNA is isolated, optionally amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in one or more of the markers described herein can be identified by hybridizing a sample and control nucleic acids, e.g., DNA or RNA, to high density arrays containing hundreds or thousands of oligonucleotides probes (Cronin et al. (1996) Human Mutation 7:244; Kozal et al. (1996) Nature Medicine 2:753). For example, genetic mutations in a marker nucleic acid can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller. specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a marker gene and detect mutations by comparing the sequence of the sample marker gene with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147).

Other methods for detecting mutations in a marker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type marker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Salceba et al. (1992) Methods Enzymol. 217:286. In one embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in marker cDNAs obtained from samples of cells. For example, the mutY enzyme of E. coli cleaves A at G/A mismatches and the thymidine DNA glycosylase from Hela cells cleaves T at G/T mismatches (Hsu et al. (1994) Carcinogenesis 15:1657). According to an exemplary embodiment, a probe based on a marker sequence, e.g., a wild-type marker sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in marker genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat. Res. 285:125; and Hayashi (1992) Genet. Anal. Tech. Appl. 9:73). Single-stranded DNA fragments of sample and control marker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant disclosure. Oligonucleotides used as primers for specific amplification may carry the mutation of interest in the center of the molecule (so that amplification depends on differential hybridization) (Gibbs et al. (1989) Nucl. Acids Res. 17:2437) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

An exemplary method for detecting the presence or absence of an analyte (e.g., DNA, RNA, protein, polypeptide, or the like) corresponding to a marker of the disclosure in a biological sample involves obtaining a bodily fluid sample (e.g., blood) from a test subject and contacting the bodily fluid sample with a compound or an agent capable of detecting one or more markers. Detection methods described herein can be used to detect one or more markers in a biological sample in vitro as well as in vivo. For example, in vitro techniques for detection of mRNA include Northern hybridizations and in situ hybridizations. In vitro techniques for detection of a polypeptide corresponding to a marker of the disclosure include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide corresponding to a marker of the disclosure include introducing into a subject a labeled antibody directed against the polypeptide. For example, the antibody can be labeled with a radioactive marker whose presence and location in a subject can be detected by standard imaging techniques. Because each marker is also an analyte, any method described herein to detect the presence or absence of a marker can also be used to detect the presence or absence of an analyte.

The markers useful in the methods of the disclosure can include any mutation in any one of the markers. Mutation sites and sequences can be identified, for example, by databases or repositories of such information, e.g., The Human Gene Mutation Database (www.hgmd.cf.ac.uk), the Single Nucleotide Polymorphism Database (dbSNP, www.ncbi.nlm.nih.gov/projects/SNP), and the Online Mendelian Inheritance in Man (OMIM) website (www.ncbi.nlm.nih.gov/omim).

The present disclosure also provides kits that comprise marker detection agents that detect at least one or more of the prostate cancer markers described herein.

The present disclosure also provides methods of treating or preventing prostate cancer in a subject comprising administering to said subject an agent that modulates the activity or expression or disrupts the function of at least one or more of the markers of this disclosure.

The one or more markers identified by this disclosure (e.g., markers in Table 1) may be used in the treatment of prostate cancer. For example, a marker (e.g., a protein or gene) identified by the present disclosure may be used as a molecular target for a therapeutic agent. A marker identified by the disclosure also may be used in any of the other methods of the disclosure, e.g., for monitoring the progression or regression of a disease or condition. In certain embodiments, the one or more markers identified by the methods of this disclosure may have therapeutic potential. For example, if a marker is identified as being up-regulated (or down-regulated), or activated (or inhibited) in phagocytic cells from a subject having prostate cancer, a compound or an agent that is capable of down-regulating (or up-regulating) or inhibiting (or activating) said marker may be useful in treating prostate cancer. Similarly, a gene protein expression level, a protein expression level, or a combination thereof may be useful in this aspect of the disclosure.

In some embodiments, a kit may be provided with reagents to measure at least two of the panel of biomarkers. The panel of biomarkers to be measured with the kit may include two or more biomarkers from the markers listed in Table 1. The kit may include reagents to measure a panel of biomarkers that includes two, three, four, five, six, seven or more biomarkers combined together to measure a subject's biomarker panel. The kit may be provided with one or more assays provided together in a kit. By way of non-limiting example, the kit may include reagents to measure the biomarkers in one assay. In some embodiments, the kit may include reagents to measure the biomarkers in more than one assay. Some kits may include a 4-plex assay and a 2-plex assay while other kits may include different combinations of assays to cover all the biomarkers needed to be measured. The kit may also include reagents to measure a biomarker individually and other biomarkers in a 2-, 4-, or 8-plex assay. Any combination of reagents and assay may be combined in a kit to cover all the biomarkers needed.

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The following examples are set forth as being representative of the present disclosure. These examples are not to be construed as limiting the scope of the disclosure as these and other equivalent embodiments will be apparent in view of the present disclosure and accompanying claims.

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein. All publications, patents, and patent applications cited herein are hereby incorporated by reference in their entirety for all purposes.

### EXAMPLES

The following examples illustrate but do not limit the compounds, compositions, and methods of the present disclosure. Other suitable modifications and adaptations of the variety of conditions and parameters normally encountered in clinical therapy and which are obvious to those skilled in the art are within the spirit and scope of the disclosure.

### Example 1

### Immunogenomic Liquid Biopsy for Prostate Cancer

This example demonstrates the use of compositions and methods disclosed herein are useful for the discovery and characterization of prostate cancer (e.g., prostate cancer signatures). In particular, that the compositions and methods disclosed are useful for generating differential transcriptomic profiles (e.g., of CD14+ and/or CD2+ cell populations) that are associated with and that can predict adverse pathologic features of prostate cancer, and that the compositions and methods disclosed herein find use in the identification, prognosis, treatment and/or management of prostate cancer patients.

### Materials and Methods

Patient population: Blood samples were collected from 1018 men who were visiting their urologist and were suspected of having prostate cancer or were known to have untreated prostate cancer and signed an informed consent to IRB approved study (WIRB #20130028).

Inclusion criteria: Men were eligible for enrollment in the study if they (i) were determined by their physician to have a risk profile that warranted a prostate biopsy, and/or (ii) had a biopsy >90 days prior to but <1 year of study entry and had not undergone definitive therapy, and/or (iii) were on active surveillance after the diagnosis of prostate cancer such that a biopsy would be performed within the next year but at least 30 days after the blood draw.

Exclusion criteria: Men were not eligible for enrollment in this study if they; i) were less than 40 years of age as prostate cancer in younger men has different behavior and characteristics, ii) had any known concurrent cancer except non-melanoma skin cancer or any history of cancer in the last 5 years, or, iii) had any form of androgen deprivation therapy (ADT) with the exception of 5-alpha reductase inhibitors.

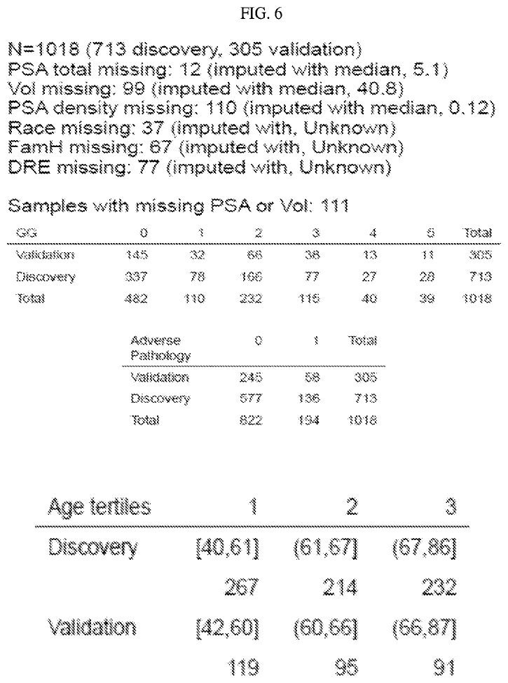

Clinical and pathological data: Clinical, laboratory, and pathology data of each patient was abstracted from the electronic medical record and entered into an electronic data capture (EDC) system by the research teams at the various institutions under the IRB approved protocol (Comprehensive Urology, Metropolitan Detroit, Michigan, Michigan Institute of Urology Metropolitan Detroit, Michigan, and Urology Austin, Austin, Texas). Pathologists at all three institutions agreed on the main standard data points to be included in the needle biopsy pathology reports. The current International Society of Urological Pathology (ISUP) modified Gleason grading system was used and the data from the highest-grade group of a single core was recorded. The maximal cross-sectional surface area of tumor on a single core and the number of positive cores were recorded in the EDC. A portion of the cases had pathology review to ensure grading uniformity between sites. For this study, the presence of adverse pathologic features was defined as any (i) Gleason grade group (GG) 4 or 5, (ii) any GG 3 with greater than 3 cores positive or greater than 30% of a core involved, or (iii) GG 2 with greater than 6 cores positive and greater than 60% of a core positive. For patients who had undergone radical prostatectomy adverse pathology was defined as GG 4 or 5 of any size, GG 3 with >30% of prostate involved, GG 3 with >10 mm tumor size, and/or GG 2 with >60% of prostate involved and with >20 mm tumor size. All available data was considered, and the most aggressive pathology was used to define cases as having adverse pathology with patients being followed up to 5 years. The demographic information of the study population is presented in FIG. 1; FIG. 6 contains details of data integrity, missing values and imputation.

Sample collection and transport: Blood samples were obtained from the three urology practices described, above. All enrolled patients signed written informed consent forms per ethical guidelines of the Institutional Review Board. Blood samples were collected in four K2EDTA BD Vacutainer™ tubes (Cat. No. 366643, BD Biosciences, San Jose, California) and transferred to the processing locations on ice at 4° C. and processed 4 hours after draw time.

CD2 and CD14 cell separation: Blood was pooled from 3 blood tubes at 4° C. and split into ⅓ and ⅔ aliquots for CD2 and CD14 cell type isolations, respectively. Specially formulated positive selection magnetic-activated cell sorting (MACS) microbeads using anti-CD2 antibodies and anti-CD14 antibodies (Cat. No. 130-101-329 and 130-101-328, respectively, Miltenyi Biotech, Bergisch Gladbach, Germany) were added to the aliquots of blood at a volume of 25 μl CD2 beads per 1 ml blood and 50 μl CD14 beads per 1 ml blood. Beads were incubated with the blood samples for 10 minutes at 4° C. The blood-bead suspensions were then processed at 4° C. using a positive selection template on the autoMACS Pro Separator (Miltenyi Biotech) to isolate the CD2 and CD14 cells. Small aliquots of the isolated CD2 and CD14 cells were removed for flow cytometry analysis while the remaining cells were pelleted by a 10-minute centrifugation at 300×g at 4° C. Following centrifugation, the supernatant was removed and 700 μL of room temperature QIAzol Lysis Reagent (Cat. No. 79306, Qiagen, Hilden, Germany) was added to each cell pellet and the cell suspension pipetted up and down for 2 minutes to lyse the cells. The suspension was then vortexed for 1 minute to further homogenize the cell lysates and frozen at −80° C.

Flow cytometry: Following their isolation, aliquots of the two white blood cell populations were stained with 1) a positive dye mix containing human CD2-FITC, human CD36-APC-Vio770, and human MC CD14 Monocyte Cocktail for staining CD2 and CD14 cells respectively, and 2) a negative dye mix consisting of human CD45-VioBlue, mouse IgG2b-FITC, mouse IgG2a-PE, mouse IgM-APC and mouse IgG2a-APC-Vio770 (Miltenyi Biotech). Only samples with purity of ≥90% for CD2 and CD14 were used.

RNA extraction: RNA extraction was accomplished using the miRNeasy Mini Kit (Cat. No. 217004, Qiagen). The frozen CD2 and CD14 cell samples (−80° C.) were thawed in a 37° C. dry bath (˜2.5 minutes) and incubated at room temperature for 5 minutes prior to the addition of 140 μL of chloroform and shaken vigorously for 15 seconds. Following a 3 min room temperature incubation, the samples were centrifuged at 12,000×g (4° C., 15 min). The upper clear aqueous phase (˜350 μL) was transferred to a 2 mL collection tube that was then placed inside the QIAcube (Cat. No. 9001292, Qiagen), and poly (A) RNA was extracted using the miRNeasy Mini Kit per manufacturer's protocol. The quality and quantity of each RNA sample was determined on a Bioanalyzer 2100 (Agilent Technologies, Santa Clara, California). Finally, the RNA samples were frozen at −80° C. and shipped to the Yale Center for Genome Analysis (YCGA) (West Haven, CT) for RNA sequencing. Only samples with high RIN (RNA Integrity Number) ≥9 were sequenced.

RNA sequencing library preparation: Samples were sent to the Yale Center for Genome Analysis (YCGA; West Haven, CT, USA) for whole transcriptome RNA sequencing. mRNA was purified from approximately 200 ng of total RNA with oligo-dT beads and sheared by incubation at 94° C. Following first-strand synthesis with random primers, second strand synthesis was performed with dUTP for generating strand-specific sequencing libraries. The cDNA library was then end-repaired, A-tailed, the adapters were ligated, and second-strand digestion was performed by uracil-DNA-glycosylase. Indexed libraries that met appropriate cut-offs for both were then quantified by qRT-PCR using a commercially available kit (KAPA Biosystems) and insert size distribution determined with the LabChip GX or Agilent Bioanalyzer. Samples with a yield of ≥0.5 ng/μL were sequenced.

Flow cell preparation and sequencing: Sample concentrations were normalized to 10 nM and loaded onto Illumina Rapid or High-output flow cells at a concentration that yields 130-250 million passing filter clusters per lane. Samples were sequenced using 75 bp paired-end sequencing on an Illumina HiSeq 2500 according to Illumina's protocols. The 6 bp index is read during an additional sequencing read that automatically follows the completion of read 1. Data generated during sequencing runs were simultaneously transferred to the YCGA high-performance computing cluster. A positive control (prepared bacteriophage Phi X library) provided by Illumina is spiked into every lane at a concentration of 0.3% to monitor sequencing quality in real time.

Sequencing data processing: Signal intensities were converted to individual base calls using the system's Real Time Analysis (RTA) software. Sample demultiplexing was performed using Illumina's CASAVA 1.8.2 software suite. Only data with sample error rate <2% and a distribution of reads per sample in a lane that is within reasonable tolerance was used. Demultiplexed raw (FASTQ) RNA sequencing data was processed using Trimmomatic for adaptor trimming, Bowtie2 for alignment to the UCSC (University of California, Santa Cruz) hg19 transcriptome, and Express for quantification. Processed reads yielded counts for 23,368 transcripts (gene symbols), corresponding to 29.8±7.5 million and 33.9±7.5 million mapped reads for CD2 and CD14 samples, respectively. Sample normalization to account for RNA concentration differences was performed using Trimmed Mean M-Value (TMM) normalization [42].

Statistical analyses: Non-normal continuous clinical covariates, namely total prostate specific antigen (PSA), prostate volume, and PSA density (PSAD) were log-transformed before further analyses. Models were built using a two-step procedure consisting of unsupervised variance-based transcript down-selection and classification by gradient boosting tree-based model (LightGBM) [43]. These models were then used to make predictions on the validation set. Models were developed on the discovery set for each cell type alone (CD2 & CD14), and for the ratio (CD14/CD2). These are considered the genomic expression only models. The performance of various clinical data (Age, Race, DRE, Family History, PSA and PSAD) was evaluated and then combined with the ratio of CD14/CD2 to investigate and determine possible enhancements to model performance.

Model performance was evaluated by determining the area under the curve (AUC) of the receiver operating characteristic (ROC) curve [44]. Differences in performance were assessed using the DeLong test [45]. All analyses were performed in R, including the gene ontology and pathway associations using the enrichR package [46-48].

### Results

Clinical and demographic characteristics of the independent training and validation sets are presented in FIG. 1. The entire cohort was collected in chronological order with the first 713 men serving as the discovery and training set, and last 315 men enrolled in the study being part of the independent validation set. While some, small differences between patients in the training and validation sets were observed for age, race, and DRE, these do not have relevant clinical implications. Patients in the training set were slightly older, somewhat more likely to be Caucasian, and had fewer abnormal DRE's. Differences in recruitment rates from the three different sites over the duration of the study most likely explain these minor differences observed in the clinicodemographic characteristics. No significant differences were observed for family history of prostate cancer, prostate volume, total PSA, PSAD, number of cancer-positive cores, maximum % of tumor involvement in a core, GG, or the adverse pathology binary endpoint. Missing data include 9.6% of prostate volume and 1.2% of PSA values leading to an overall 10.8% of cases where PSA density could not be calculated (See FIG. 6). The worst pathology at any time during follow-up was used to define the patients' status, in particular for the binary, adverse pathology endpoint, with a median follow-up of 3.8 years (interquartile range: 1.2-4.7 years) for the patients enrolled in this study.

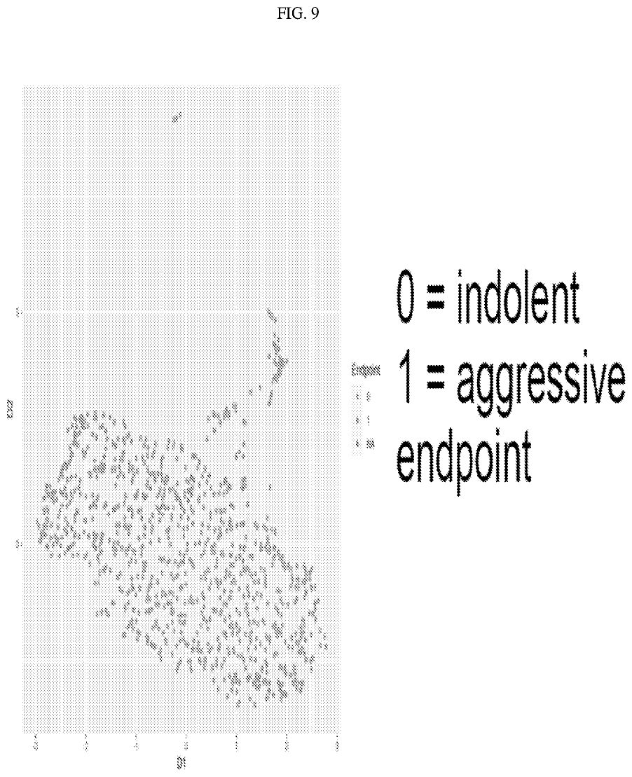

Initial filtering of the transcriptomic data resulted in a reduced set of 18,703 transcripts with observed expression (nonzero counts) in at least 15% of the samples in either CD2 or CD14. While log-transformed CD14 and CD2 counts were analyzed separately, the CD2-normalized CD14 signal was also used as model input. The normalization comprised subtracting the log-transformed CD2 counts from the log-transformed CD14 counts per patient, on a gene-by-gene basis. This is also referred to as the log (CD14/CD2) ratio. Exploratory principal component analysis on log-transformed ratios of CD2 and CD14 data, log (CD14/CD2), revealed no significant batch effects between the training and validation datasets (See FIG. 9).

The clinico-genomic model is built on a two-step procedure comprising transcript down-selection and classification. For the former, (unsupervised) variance-based transcript selection was applied using the training set (N=713). This down-selection stage was used to prevent the model from overfitting because the number of transcripts (17,138) is much larger than the number of subjects (N=713), which is a well-recognized issue in regularized models when the effect sizes of individual transcripts are small. In the second step, age, log-transformed total PSA, log-transformed PSAD, and the transcripts selected by variance as inputs to the model were considered. To optimize the hyperparameters, namely, the number of transcripts selected by variance and the regularization parameter of the gradient boosting tree-based model used as classifier (LightGBM) model, 10-fold cross-validation on the training set was used. Parameters of LightGBM other than the regularization strength were set to their default values. LightGBM is a gradient boosting machine (GBM) algorithm that combines (ensembles) the predictions of a collection of decision trees, each of which, considers a subset of model inputs, thus often resulting in performance improvements relative to standard approaches such as logistic regression. Small variations of these hyperparameters were verified to not substantially change the performance of the model. The final model is built on the entire training set restricted to the selected transcripts and the optimal regularization parameter found by cross-validation. This model was then used to make predictions on the validation set.

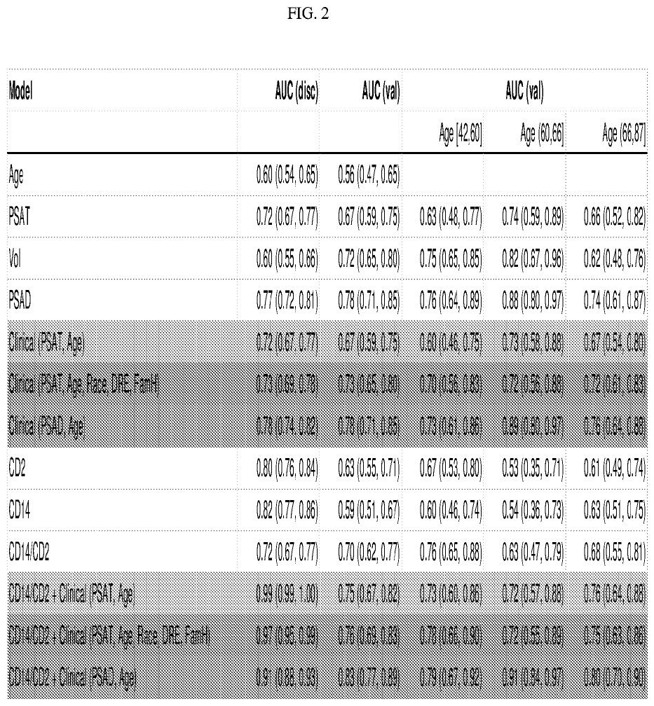

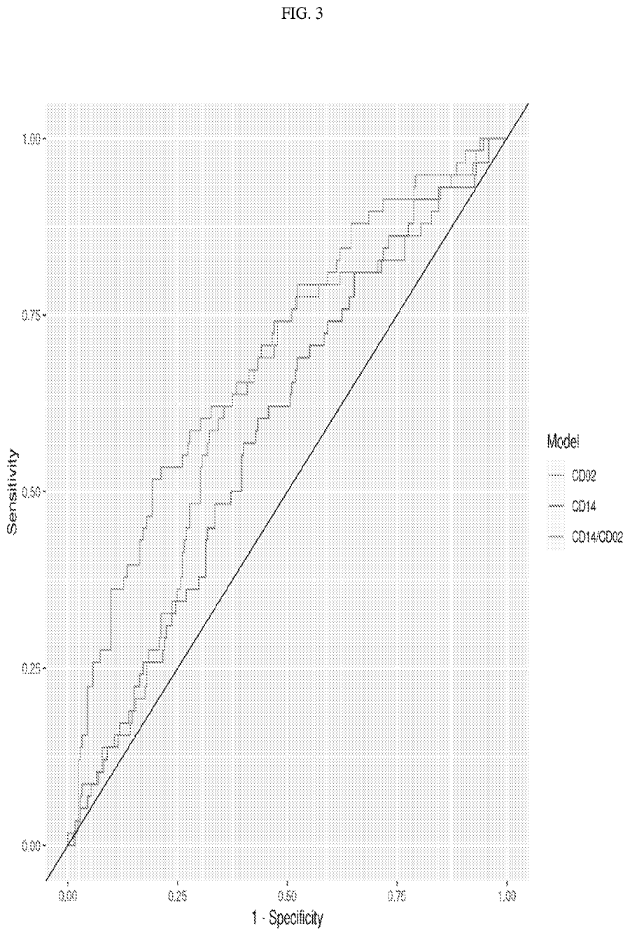

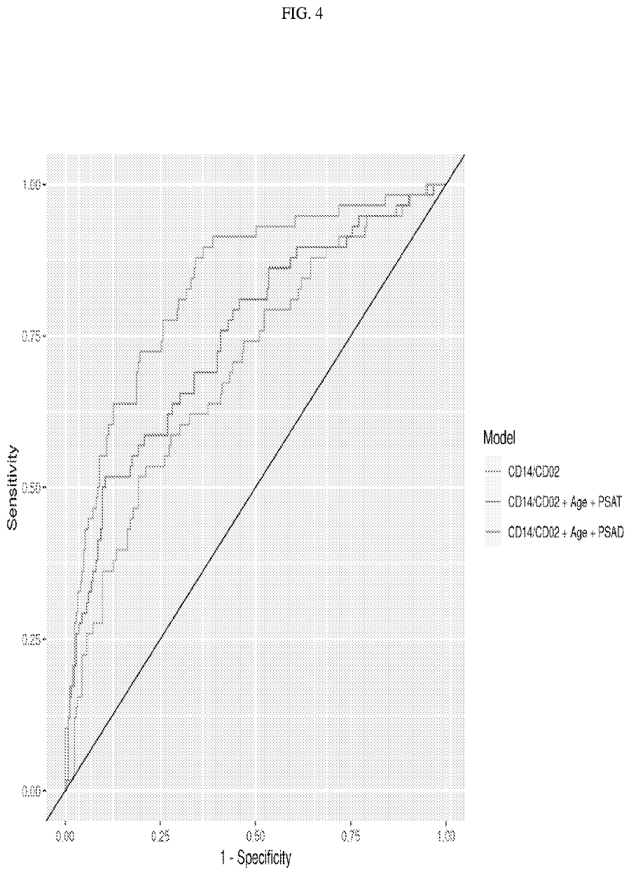

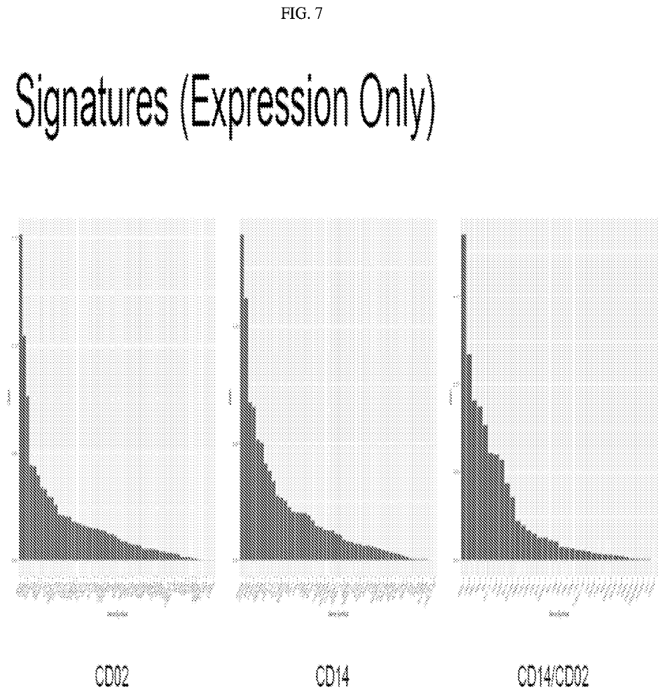

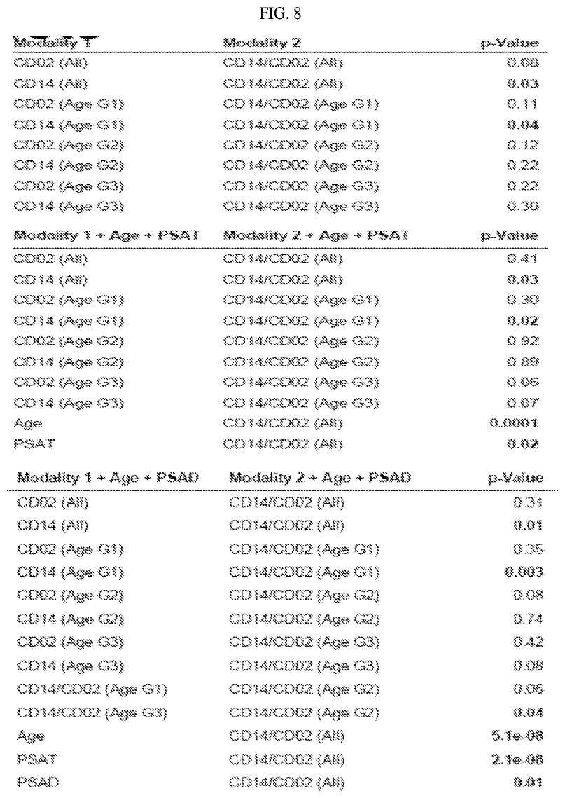

Using this two-step procedure, transcriptomic models were built based on either the individual cell type counts for CD2 and CD14, but also using the CD14/CD2 ratio (FIG. 2). The genes and weighting factors of these models are shown in FIG. 7. The data demonstrates that the best performing immunotranscriptomic model, is the one based on the CD14/CD2 ratio, emphasizing both the tumor phagocytosis mechanism and the anti-tumor immune response, yielded an AUC of 0.70. This compares favorably to the CD14 and CD2 only models, which resulted in AUCs of 0.59 (p=0.033) and 0.63 (p=0.079), respectively, indicating that the ratio has significantly increased performance in predicting adverse pathology over either cell type alone (FIG. 3). Note that since the ratio (i) is taken on a log scale it is considered as a form of subtraction of underlying background within the immune system (reducing noise), and (ii) outperformed both individual CD14 and CD2 modalities, it was used as the basic immunotranscriptomic component for more advanced modeling exercises.

The same LightGBM optimization procedure was also applied to combine immunotranscriptomics with readily available clinical and demographic risk factors. This resulted in increased performance in predicting adverse pathology compared to immunotranscriptomics alone. An immunotranscriptomics model combined with the simplest clinical risk factors, PSA and age, significantly (p=0.02) outperformed these same clinical risk factors alone. While a measurement of prostate volume is not always readily available, it is known to be a significant risk factor in detecting adverse pathology and a reliable aid in the management of prostate cancer patients. When available, prostate volume is typically used to normalize PSA levels, resulting in a metric called PSAD (PSA/prostate volume). A model combining immunotranscriptomics with PSAD and age reached an AUC of 0.83 in the independent validation set (FIG. 2), a significant improvement compared to a model based on age and PSA density alone, which yielded an AUC of 0.78 (p.=0.01).

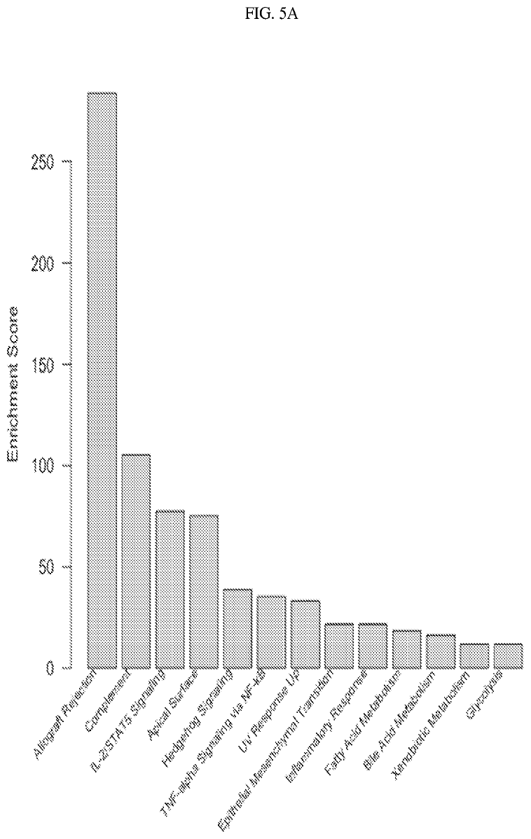

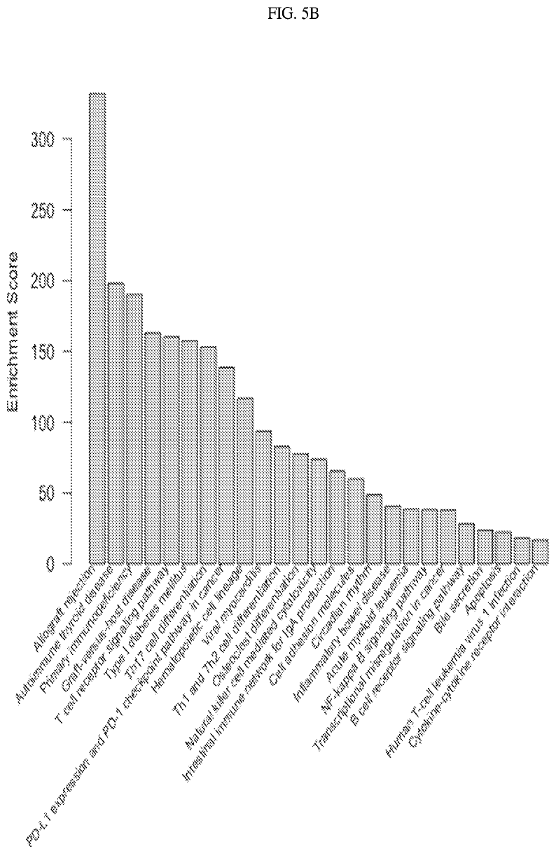

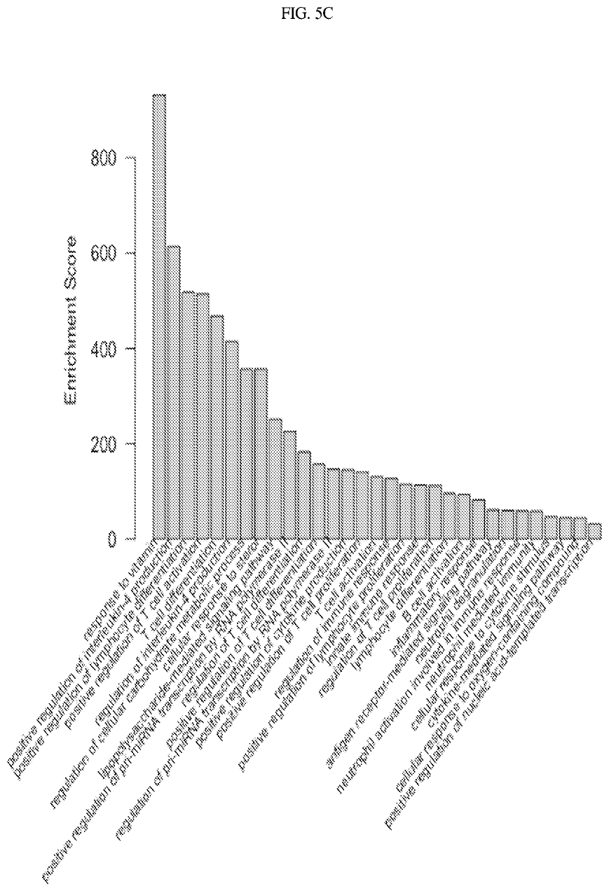

The biological relevance of the genes involved in the best performing model, i.e. immune-transcriptomics, age, and PSAD, was further evaluated through pathway and ontology analysis. This model included a set of 120 genes (Table 1). Immune response and immune system related pathways were significantly enriched (false discovery rate <0.1) within this set of genes, as evidenced in the hallmarks MSigDB database (FIG. 5A), KEGG database (FIG. 5B), and gene ontology biological processes (FIG. 5C). Interestingly, several cancer-related pathways also appear to be significantly over-represented by this geneset, most notably, hedgehog signaling (FIG. 5A), epithelial mesenchymal transition (FIG. 5A), PDL1 and PD1 checkpoint (FIG. 5B), and transcriptional misregulation in cancer (FIG. 5B). Other pathways can be linked to either the general function, response, or activation of phagocytic immune cells, or the presence of cancer cells, e.g. TNF alpha and NF-kappa B signaling, acute myeloid leukemia, and apoptosis. However, due to the nature of the gene selection procedure with the gradient boosting method, selecting for independent contributors, significant enrichment of certain pathways was not necessarily expected. Indeed, in some aspects, the set of 120 genes link to phagocytic activity and cancer-related genes in KEGG. The phagocytic component is clearly represented by Fc gamma R-mediated phagocytosis and phagosome. In addition to the general pathways in cancer, choline metabolism in cancer, colorectal/breast/gastric cancer, microRNAs in cancer, proteoglycans in cancer appear in the list of KEGG-terms associated with the set of 120 genes included in the final model (FIG. 5).

In order to further explore the effect of patient age on the model performance, the analysis was also done by stratifying into age groups. This did not show statistically significant differences for most age group dependent models. The model including PSAD and age together with immunotranscriptomics did show a trend toward better performance in the 60-66 year age range with an AUC of 0.91 in the independent validation set, compared to the 42-60 year age range with an AUC of 0.79 (p=0.06), and a significant difference in the 66-87 age range with an AUC of 0.80 (p=0.04).

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in relevant fields are intended to be within the scope of the following claims.

### REFERENCES

1. Hou J S, He Y Y, Du L L, Duan Z M, Chen X, Li M. Gene expression profile and long noncoding RNA analysis in Candida albicans insoluble β-glucan-stimulated CD14+ monocytes and THP-1 cells. Microb Pathog. 2021 May 20; 157:104963. doi: 10.1016/j.micpath.2021.104963. Epub ahead of print. PMID: 34022361.
2. Reynolds L M, Ding J, Taylor J R, Lohman K, Soranzo N, de la Fuente A, Liu T F. Johnson C, Barr R G, Register T C, Donohue K M, Talor M V, Cihakova D, Gu C, Divers J, Siscovick D, Burke G, Post W, Shea S, Jacobs D R Jr, Hoeschele I, McCall C E, Kritchevsky S B, Herrington D. Tracy R P, Liu Y. Transcriptomic profiles of aging in purified human immune cells. BMC Genomics. 2015 Apr. 22; 16 (1): 333. doi: 10.1186/s12864-015-1522-4. PMID: 25898983; PMCID: PMC4417516.
3. Ruiz-Limon P, Ortega-Castro R. Barbarroja N, Perez-Sanchez C, Jamin C, Patiño-Trives A M, Luque-Tevar M, Ibáñez-Costa A, Perez-Sanchez L, de la Rosa I A, Abalos-Aguilera M, Jimenez-Gomez Y, Calvo-Gutierrez J. Font P. Escudero-Contreras A, Alarcon-Riquelme M E, Collantes-Estevez E, López-Pedrera C; PRECISESADS Clinical Consortium and Flow Cytometry Study Group. Molecular Characterization of Monocyte Subsets Reveals Specific and Distinctive Molecular Signatures Associated With Cardiovascular Disease in Rheumatoid Arthritis. Front Immunol. 2019 May 21; 10:1111. doi: 10.3389/fimmu.2019.01111. PMID: 31169830; PMCID: PMC6536567.
4. Wang L, Felts S J, Van Keulen V P, Scheid A D, Block M S, Markovic S N, Pease L R, Zhang Y. Integrative Genome-Wide Analysis of Long Noncoding RNAs in Diverse Immune Cell Types of Melanoma Patients. Cancer Res. 2018 Aug. 1; 78 (15): 4411-4423. doi: 10.1158/0008-5472.CAN-18-0529. Epub 2018 Jun. 12. PMID: 29895674; PMCID: PMC6072578.
5. Palmer C, Dichn M, Alizadeh A A, Brown P O. Cell-type specific gene expression profiles of leukocytes in human peripheral blood. BMC Genomics. 2006 May 16; 7:115. doi: 10.1186/1471-2164-7-115. PMID: 16704732; PMCID: PMC1479811.
6. Itoh K, Okubo K, Utiyama H, Hirano T, Yoshii J, Matsubara K. Expression profile of active genes in granulocytes. Blood. 1998 Aug. 15; 92 (4): 1432-41. PMID: 9694733.
7. Hashimoto S, Nagai S, Sese J, Suzuki T, Obata A, Sato T, Toyoda N, Dong H Y, Kurachi M. Nagahata T, Shizuno K, Morishita S, Matsushima K. Gene expression profile in human leukocytes. Blood. 2003 May 1; 101 (9): 3509-13. doi: 10.1182/blood-20Feb. 6, 1866. Epub 2003 Jan. 9. PMID: 12522010.
8. Allantaz F. Cheng D T, Bergauer T. Ravindran P. Rossier M F, Ebeling M, Badi L, Reis B, Bitter H, D'Asaro M, Chiappe A, Sridhar S, Pacheco G D, Burczynski M E, Hochstrasser D. Vonderscher J, Matthes T. Expression profiling of human immune cell subsets identifies miRNA-mRNA regulatory relationships correlated with cell type specific expression. PLOS One. 2012; 7 (1): c29979. doi: 10.1371/journal.pone.0029979. Epub 2012 Jan. 20. PMID: 22276136; PMCID: PMC3262799.
9. Ross R W, Galsky M D, Scher H I, Magidson J, Wassmann K, Lec G S, Katz L, Subudhi S K. Anand A, Fleisher M, Kantoff P W, Oh W K. A whole-blood RNA transcript-based prognostic model in men with castration-resistant prostate cancer: a prospective study. Lancet Oncol. 2012 November; 13 (11): 1105-13. doi: 10.1016/S1470-2045 (12) 70263-2. Epub 2012 Oct. 9. PMID: 23059047.
10. Olmos D, Brewer D. Clark J, Danila D C, Parker C, Attard G, Fleisher M, Reid A H, Castro E, Sandhu S K. Barwell L, Oommen N B, Carreira S, Drake C G, Jones R, Cooper C S, Scher H I, de Bono J S. Prognostic value of blood mRNA expression signatures in castration-resistant prostate cancer: a prospective, two-stage study. Lancet Oncol. 2012 November; 13 (11): 1114-24. doi: 10.1016/S1470-2045 (12) 70372-8. Epub 2012 Oct. 9. PMID: 23059046; PMCID: PMC4878433.
11. Wang L. Gong Y, Chippada-Venkata U, Heck M M, Retz M, Nawroth R, Galsky M, Tsao C K, Schadt E, de Bono J. Olmos D, Zhu J, Oh W K. A robust blood gene expression-based prognostic model for castration-resistant prostate cancer. BMC Med. 2015 Aug. 21; 13:201. doi: 10.1186/s12916-015-0442-0. PMID: 26297150; PMCID: PMC4546313.
12. Huen N Y, Pang A L, Tucker J A, Lee T L, Vergati M, Jochems C, Intrivici C, Cereda V, Chan W Y. Rennert O M, Madan R A, Gulley J L, Schlom J. Tsang K Y. Up-regulation of proliferative and migratory genes in regulatory T cells from patients with metastatic castration-resistant prostate cancer. Int J Cancer. 2013 Jul. 15; 133 (2): 373-82. doi: 10.1002/ijc.28026. Epub 2013 Feb. 12. PMID: 23319273; PMCID: PMC3695702.
13. Wallace T A, Downey R F, Seufert C J, Schetter A, Dorsey T H, Johnson C A, Goldman R, Loffredo C A. Yan P. Sullivan F J, Giles F J, Wang-Johanning F, Ambs S, Glynn S A. Elevated HERV-K mRNA expression in PBMC is associated with a prostate cancer diagnosis particularly in older men and smokers. Carcinogenesis. 2014 September; 35 (9): 2074-83. doi: 10.1093/carcin/bgu114. Epub 2014 May 23. PMID: 24858205; PMCID: PMC4146419.
14. Showe M K, Vachani A, Kossenkov A V, Yousef M, Nichols C, Nikonova E V, Chang C. Kucharczuk J, Tran B, Wakcam E, Yic T A, Speicher D, Rom W N, Albelda S, Showe L C. Gene expression profiles in peripheral blood mononuclear cells can distinguish patients with non-small cell lung cancer from patients with nonmalignant lung disease. Cancer Res. 2009 Dec. 15; 69 (24): 9202-10. doi: 10.1158/0008-5472.CAN-09-1378. PMID: 19951989; PMCID: PMC2798582.
15. Zander T. Hofmann A, Staratschek-Jox A, Classen S, Debey-Pascher S, Maisel D, Ansén S, Hahn M, Beyer M, Thomas R K, Gathof B, Mauch C, Delank K S, Engel-Riedel W. Wichmann H E, Stoelben E. Schultze J L, Wolf J. Blood-based gene expression signatures in non-small cell lung cancer. Clin Cancer Res. 2011 May 15; 17 (10): 3360-7. doi: 10.1158/1078-0432.CCR-10-0533. Epub 2011 May 10. PMID: 21558400.
16. Kossenkov A V, Dawany N, Evans T L, Kucharczuk J C, Albelda S M, Showe L C, Showe M K. Vachani A. Peripheral immune cell gene expression predicts survival of patients with non-small cell lung cancer. PLOS One. 2012; 7 (3): e34392. doi: 10.1371/journal.pone.0034392. Epub 2012 Mar. 29. PMID: 22479623; PMCID: PMC3315526.
17. Ma J, Lin Y, Zhan M, Mann D L, Stass S A, Jiang F. Differential miRNA expressions in peripheral blood mononuclear cells for diagnosis of lung cancer. Lab Invest. 2015 October; 95 (10): 1197-206. doi: 10.1038/labinvest.2015.88. Epub 2015 Jul. 6. PMID: 26146958; PMCID: PMC4586315.
18. Kossenkov A V, Qureshi R, Dawany N B, Wickramasinghe J, Liu Q. Majumdar R S, Chang C, Widura S, Kumar T, Horng W H, Konnisto E, Criner G. Tsay J J, Pass H, Yendamuri S, Vachani A, Bauer T, Nam B, Rom W N, Showe M K, Showe L C. A Gene Expression Classifier from Whole Blood Distinguishes Benign from Malignant Lung Nodules Detected by Low-Dose C T. Cancer Res. 2019 Jan. 1; 79 (1): 263-273. doi: 10.1158/0008-5472.CAN-18-2032. Epub 2018 November 28. PMID: 30487137; PMCID: PMC6317999.
19. Baine M J, Menning M, Smith L M, Mallya K, Kaur S, Rachagani S. Chakraborty S, Sasson A R. Brand R E, Batra S K. Differential gene expression analysis of peripheral blood mononuclear cells reveals novel test for early detection of pancreatic cancer. Cancer Biomark. 2011-2012; 11 (1): 1-14. doi: 10.3233/CBM-2012-0260. PMID: 22820136; PMCID: PMC3557848.
20. Batliwalla F M, Bacchler E C, Xiao X. Li W. Balasubramanian S, Khalili H, Damle A, Ortmann W A, Perrone A, Kantor A B, Gulko P S, Kern M, Furie R, Behrens T W, Gregersen P K. Peripheral blood gene expression profiling in rheumatoid arthritis. Genes Immun. 2005 August; 6 (5): 388-97. doi: 10.1038/sj.gene.6364209. PMID: 15973463.
21. Ramilo O, Allman W, Chung W, Mejias A, Ardura M, Glaser C, Wittkowski K M, Piqueras B, Banchereau J, Palucka A K, Chaussabel D. Gene expression patterns in blood leukocytes discriminate patients with acute infections. Blood. 2007 Mar. 1; 109 (5): 2066-77. doi: 10.1182/blood-2006-02-002477. Epub 2006 Nov. 14. PMID: 17105821; PMCID: PMC1801073.
22 Dale D C, Boxer L, Liles W C. The phagocytes: neutrophils and monocytes. Blood. 2008 Aug. 15; 112 (4): 935-45. doi: 10.1182/blood-2007-12-077917. PMID: 18684880.
23. Chow A. Brown B D, Merad M. Studying the mononuclear phagocyte system in the molecular age. Nat Rev Immunol. 2011 Oct. 25; 11 (11): 788-98. doi: 10.1038/nri3087. PMID: 22025056.
24. Gutknecht M F, Bouton A H. Functional significance of mononuclear phagocyte populations generated through adult hematopoiesis. J Leukoc Biol. 2014 December; 96 (6): 969-80. doi: 10.1189/jlb.1RI0414-195R. Epub 2014 Sep. 15. PMID: 25225678; PMCID: PMC4226790.
25. Pritchard C C, Mateo J, Walsh M F, De Sarkar N, Abida W, Beltran H, Garofalo A, Gulati R, Carreira S, Eeles R, Elemento O, Rubin M A, Robinson D, Lonigro R. Hussain M, Chinnaiyan A. Vinson J, Filipenko J, Garraway L. Taplin M E, AlDubayan S. Han G C, Beightol M. Morrissey C, Nghiem B, Cheng H H, Montgomery B, Walsh T, Casadei S, Berger M, Zhang L, Zehir A, Vijai J, Scher H I, Sawyers C, Schultz N, Kantoff P W, Solit D, Robson M, Van Allen E M, Offit K, de Bono J, Nelson P S. Inherited DNA-Repair Gene Mutations in Men with Metastatic Prostate Cancer. N Engl J Med. 2016 Aug. 4; 375 (5): 443-53. doi: 10.1056/NEJMoa1603144. Epub 2016 Jul. 6. PMID: 27433846; PMCID: PMC4986616.
26. Wang G, Zhao D, Spring D J, DePinho R A. Genetics and biology of prostate cancer. Genes Dev. 2018 Sep. 1; 32 (17-18): 1105-1140. doi: 10.1101/gad.315739.118. PMID: 30181359; PMCID: PMC6120714.
27. Ross-Adams H, Lamb A D, Dunning M J, Halim S, Lindberg J, Massic C M, Egevad L A, Russell R. Ramos-Montoya A, Vowler S L, Sharma N L, Kay J, Whitaker H, Clark J, Hurst R. Gnanapragasam V J, Shah N C, Warren A Y, Cooper C S, Lynch A G, Stark R. Mills I G, Grönberg H, Neal D E; CamCaP Study Group. Integration of copy number and transcriptomics provides risk stratification in prostate cancer: A discovery and validation cohort study. EBioMedicine. 2015 Jul. 29; 2 (9): 1133-44. doi: 10.1016/j.ebiom.2015.07.017. Erratum in: EBioMedicine. 2017 March; 17:238. PMID: 26501111; PMCID: PMC4588396.
28. Wang G, Xu D, Zhang Z, Li X, Shi J, Sun J, Liu H Z, Li X, Zhou M, Zheng T. The pan-cancer landscape of crosstalk between epithelial-mesenchymal transition and immune evasion relevant to prognosis and immunotherapy response. NPJ Precis Oncol. 2021 Jun. 22; 5 (1): 56. doi: 10.1038/s41698-021-00200-4. PMID: 34158591.

29 Efremova M, Rieder D. Klepsch V, Charoentong P, Finotello F, Hackl H, Hermann-Kleiter N, Löwer M, Baier G, Krogsdam A. Trajanoski Z. Targeting immune checkpoints potentiates immunoediting and changes the dynamics of tumor evolution. Nat Commun. 2018 Jan. 2; 9 (1): 32. doi: 10.1038/s41467-017-02424-0. PMID: 29296022; PMCID: PMC5750210.
30. Dunn G P, Bruce A T, Ikeda H, Old L J, Schreiber R D. Cancer immunoediting: from immunosurveillance to tumor escape. Nat Immunol. 2002 November; 3 (11): 991-8. doi: 10.1038/ni1102-991. PMID: 12407406.
31. Gasser S. Raulet D H. The DNA damage response arouses the immune system. Cancer Res. 2006 Apr. 15; 66 (8): 3959-62. doi: 10.1158/0008-5472.CAN-05-4603. PMID: 16618710.
32. “Immune Surveillance Gert Jan Fleuren, . . . . Peter J. K. Kuppen, in Encyclopedia of Immunology (Second Edition), 1998.” Encyclopedia of Immunology, by Peter Delves and Ivan Roitt, Academic Press, 1998.
33. Murphy, Kenneth, et al. Janeway's Immunobiology. Garland Science, 2012.
34. Aderem A, Underhill D M. Mechanisms of phagocytosis in macrophages. Annu Rev Immunol. 1999; 17:593-623. doi: 10.1146/annurev.immunol.17.1.593. PMID: 10358769.
35. Foulds G A, Vadakekolathu J, Abdel-Fatah T M A, Nagarajan D, Recder S, Johnson C, Hood S, Moseley P M, Chan S Y T, Pockley A G, Rutella S, McArdle S E B. Immune-Phenotyping and Transcriptomic Profiling of Peripheral Blood Mononuclear Cells From Patients With Breast Cancer: Identification of a 3 Gene Signature Which Predicts Relapse of Triple Negative Breast Cancer. Front Immunol. 2018 Sep. 11; 9:2028. doi: 10.3389/fimmu.2018.02028. PMID: 30254632; PMCID: PMC6141692.
36. Shill D K, Roobol M J, Ehdaie B, Vickers A J, Carlsson S V. Active surveillance for prostate cancer. Transl Androl Urol. 2021 June; 10 (6): 2809-2819. doi: 10.21037/tau-20-1370. PMID: 34295763; PMCID: PMC8261451.

37 Kaul S, Wojno K J, Stone S, Evans B, Bernhisel R, Meek S, D'Anna R E, Ferguson J, Glaser J, Morgan T M, Lieb J, Yan R, Cohen T, Ehdaie B. Clinical outcomes in men with prostate cancer who selected active surveillance using a clinical cell cycle risk score. Per Med. 2019 November; 16 (6): 491-499. doi: 10.2217/pme-2019-0084. Epub 2019 Sep. 4. Erratum in: Per Med. 2020 November; 17 (6): 491. PMID: 31483217; PMCID: PMC7447833.
38. Egevad L, Delahunt B, Srigley J R, Samaratunga H. International Society of Urological Pathology (ISUP) grading of prostate cancer—An ISUP consensus on contemporary grading. APMIS. 2016 June; 124 (6): 433-5. doi: 10.1111/apm.12533. PMID: 27150257.
39. Bolger A M, Lohse M, Usadel B. Trimmomatic: a flexible trimmer for Illumina sequence data. Bioinformatics. 2014 Aug. 1; 30 (15): 2114-20. doi: 10.1093/bioinformatics/btu170. Epub 2014 Apr. 1. PMID: 24695404; PMCID: PMC4103590.
40. Langmead B, Salzberg S L. Fast gapped-read alignment with Bowtie 2. Nat Methods. 2012 Mar. 4; 9 (4): 357-9. doi: 10.1038/nmeth.1923. PMID: 22388286; PMCID: PMC3322381.
41. Roberts A, Pachter L. Streaming fragment assignment for real-time analysis of sequencing experiments. Nat Methods. 2013 January; 10 (1): 71-3. doi: 10.1038/nmeth.2251. Epub 2012 Nov. 18. PMID: 23160280; PMCID: PMC3880119.
42. Robinson M D, Oshlack A. A scaling normalization method for differential expression analysis of RNA-seq data. Genome Biol. 2010; 11 (3): R25. doi: 10.1186/gb-2010-11-3-r25. Epub 2010 Mar. 2. PMID: 20196867; PMCID: PMC2864565.
43. Ke G, Meng Q, Finley T. Wang T, Chen W, Ma W. Ye Q. Liu T Y. Lightgbm: A highly efficient gradient boosting decision tree. Advances in neural information processing systems. 2017; 30:3146-54.
44. Fawcett T. An introduction to ROC analysis. Pattern recognition letters. 2006 Jun. 1; 27 (8): 861-74.
45. DeLong E R, DeLong D M, Clarke-Pearson D L. Comparing the areas under two or more correlated receiver operating characteristic curves: a nonparametric approach. Biometrics. 1988 September; 44 (3): 837-45. PMID: 3203132.
46. Chen E Y, Tan C M, Kou Y, Duan Q. Wang Z. Meirelles G V, Clark N R, Ma'ayan A. Enrichr: interactive and collaborative HTML5 gene list enrichment analysis tool. BMC Bioinformatics. 2013; 128 (14).
47. Kuleshov M V. Jones M R, Rouillard A D, Fernandez N F, Duan Q. Wang Z. Koplev S, Jenkins S L, Jagodnik K M, Lachmann A, McDermott M G, Monteiro C D, Gundersen G W, Ma'ayan A. Enrichr: a comprehensive gene set enrichment analysis web server 2016 update. Nucleic Acids Research. 2016; gkw377.
48. Xie Z, Bailey A, Kuleshov M V, Clarke D J B., Evangelista J E, Jenkins S L, Lachmann A, Wojciechowicz M L, Kropiwnicki E, Jagodnik K M, Jeon M, & Ma'ayan A. Gene set knowledge discovery with Enrichr. Current Protocols, 1, e90. 2021. doi: 10.1002/cpz1.90

1-54. (canceled)

55. A method for detecting the presence or absence of prostate cancer in a subject, the method comprising: obtaining a biological sample from the subject, wherein the biological sample comprises CD2+ cells and CD14+ cells;
measuring the gene expression level of five or more biomarkers selected from CACNA1I, PDK4, TREML4, ODZ1, COL5A3, SLC11A1, GZMM, LOC283174, KRT72, SLC4A10, LBH, CHST15, COL6A2, ZNF683, ALPL, CSF1R, SCGB3A1, POM121L1P, SAMD3, FAM198B, NEBL, RASGRP1, KYNU, RTN1, CTSW, RORC, ABCB1, LMO2, S100A12, CD27, SH2D1A, PDZD4, CD300LB, FPR2, CLEC4F, AQP3, FOS, PTCH1, ASGR2, MARCKS, SIGLEC14, EGR1, MPEG1, SYNE2, POM121L4P, CD40LG, PYGL, MYCL1, CSMD1, BACH2, ENO2, LOXHD1, LILRA1, SLC44A5, CCDC64, IGSF9B, RORA, CD6, SCML4, RHOH, VCAN, CXCR3, PID1, EGR2, PRF1, NFAM1, SAPCD2, ADAMTSL4, TARP, PYHIN1, SKAP1, SPI1, MAL, KLRB1, KRT1, B3GAT1, ANPEP, CD86, MS4A14, FGFBP2, MS4A7, GPR15, CCR4, F13A1, CD247, EOMES, CD1D, C19orf59, LOC284837, TC2N, LCK, GZMB, GZMA, NAPSB, ZAP70, ALDH1A1, CD28, HIPIR, GPR56, S100A9, CLEC12A, GATA3, CD5, CST7, CLEC4E, TIGIT, HCK, ATP1A4, AQP9, GZMH, SULF2, LILRA3, CD14, IL7R, RIN2, VNN1, NCALD, TREM1, SYCP2L, and LGALS2 in the subjects' CD14+ cells;
measuring the gene expression level of the five or more selected markers in the subject's CD2+ cells; and
identifying the difference between the measured levels of the five or more selected markers in order to determine the ratio of CD14+/CD2+ expression;
wherein the ratio of CD14+/CD2+ expression indicates the presence or absence of prostate cancer in the subject.

56. The method of claim 55, comprising measuring the gene expression level of ten or more biomarkers selected from CACNA1I, PDK4, TREML4, ODZ1, COL5A3, SLC11A1, GZMM, LOC283174, KRT72, SLC4A10, LBH, CHST15, COL6A2, ZNF683, ALPL, CSF1R, SCGB3A1, POM121LIP, SAMD3, FAM198B, NEBL, RASGRP1, KYNU, RTN1, CTSW, RORC, ABCB1, LMO2, S100A12, CD27, SH2D1A, PDZD4, CD300LB, FPR2, CLEC4F, AQP3, FOS, PTCH1, ASGR2, MARCKS, SIGLEC14, EGR1, MPEG1, SYNE2, POM121L4P, CD40LG, PYGL, MYCL1, CSMD1, BACH2, ENO2, LOXHD1, LILRA1, SLC44A5, CCDC64, IGSF9B, RORA, CD6, SCML4, RHOH, VCAN, CXCR3, PID1, EGR2, PRF1, NFAM1, SAPCD2, ADAMTSL4, TARP, PYHIN1, SKAP1, SPI1, MAL, KLRB1, KRT1, B3GAT1, ANPEP, CD86, MS4A14, FGFBP2, MS4A7, GPR15, CCR4, F13A1, CD247, EOMES, CD1D, C19orf59, LOC284837, TC2N, LCK, GZMB, GZMA, NAPSB, ZAP70, ALDH1A1, CD28, HIPIR, GPR56, S100A9, CLEC12A, GATA3, CD5, CST7, CLEC4E, TIGIT, HCK, ATP1A4, AQP9, GZMH, SULF2, LILRA3, CD14, IL7R, RIN2, VNN1, NCALD, TREM1, SYCP2L, and LGALS2.

57. The method of claim 55, comprising measuring the gene expression level of twenty or more biomarkers selected from CACNA1I, PDK4, TREML4, ODZ1, COL5A3, SLC11A1, GZMM, LOC283174, KRT72, SLC4A10, LBH, CHST15, COL6A2, ZNF683, ALPL, CSF1R, SCGB3A1, POM121L1P, SAMD3, FAM198B, NEBL, RASGRP1, KYNU, RTN1, CTSW, RORC, ABCB1, LMO2, S100A12, CD27, SH2D1A, PDZD4, CD300LB, FPR2, CLEC4F, AQP3, FOS, PTCH1, ASGR2, MARCKS, SIGLEC14, EGR1, MPEG1, SYNE2, POM121L4P, CD40LG, PYGL, MYCL1, CSMD1, BACH2, ENO2, LOXHD1, LILRA1, SLC44A5, CCDC64, IGSF9B, RORA, CD6, SCML4, RHOH, VCAN, CXCR3, PID1, EGR2, PRF1, NFAM1, SAPCD2, ADAMTSL4, TARP, PYHIN1, SKAP1, SPI1, MAL, KLRB1, KRT1, B3GAT1, ANPEP, CD86, MS4A14, FGFBP2, MS4A7, GPR15, CCR4, F13A1, CD247, EOMES, CD1D, C19orf59, LOC284837, TC2N, LCK, GZMB, GZMA, NAPSB, ZAP70, ALDH1A1, CD28, HIPIR, GPR56, S100A9, CLEC12A, GATA3, CD5, CST7, CLEC4E, TIGIT, HCK, ATP1A4, AQP9, GZMH, SULF2, LILRA3, CD14, IL7R, RIN2, VNN1, NCALD, TREM1, SYCP2L, and LGALS2.

58. The method according to claim 55, further comprising measuring at least one standard parameter associated with prostate cancer.

59. The method of claim 58, wherein the standard parameter is selected from the group consisting of tumor stage, tumor grade, tumor size, tumor visual characteristics, tumor growth, tumor thickness, tumor progression, tumor metastasis tumor distribution within the body, odor, molecular pathology, genomics, or tumor angiograms.

60. The method according to claim 55, further comprising obtaining one or more clinical data from the subject selected from the group consisting of age, race, digital rectal exam (DRE), prostate volume, prostate density, total prostate-specific antigen (PSA), and PSA density.

61. The method of claim 60, wherein the one or more clinical data are used as clinical covariates and concatenated with the ratio of CD14+/CD2+ expression and input into a sparse rank regression model.

62. The method of claim 55, wherein the gene expression levels are measured by polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpalI tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, methyl-binding PCR analysis, or a combination thereof.

63. The method of claim 55, wherein the gene expression levels are measured by a sequencing technique selected from the group consisting of direct sequencing, RNA sequencing, whole transcriptome shotgun sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and a combination thereof.

64. The method of claim 55, wherein the subject is a human.

65. The method of claim 55, wherein the CD14+ cells are isolated from a bodily fluid sample, tissues, or cells of the subject.

66. The method of claim 55, wherein the CD2+ cells are isolated from a bodily fluid sample, tissues, or cells of the subject.

67. The method of claim 65, wherein the bodily fluid sample is blood, urine, stool, saliva, lymph fluid, cerebrospinal fluid, synovial fluid, cystic fluid, ascites, pleural effusion, fluid obtained from a pregnant woman in the first trimester, fluid obtained from a pregnant woman in the second trimester, fluid obtained from a pregnant woman in the third trimester, maternal blood, amniotic fluid, chorionic villus sample, fluid from a preimplantation embryo, maternal urine, maternal saliva, placental sample, fetal blood, lavage and cervical vaginal fluid, interstitial fluid, or ocular fluid.

68. The method of claim 66, wherein the bodily fluid sample is blood, urine, stool, saliva, lymph fluid, cerebrospinal fluid, synovial fluid, cystic fluid, ascites, pleural effusion, fluid obtained from a pregnant woman in the first trimester, fluid obtained from a pregnant woman in the second trimester, fluid obtained from a pregnant woman in the third trimester, maternal blood, amniotic fluid, chorionic villus sample, fluid from a preimplantation embryo, maternal urine, maternal saliva, placental sample, fetal blood, lavage and cervical vaginal fluid, interstitial fluid, or ocular fluid.
